(19) United States
(12) Patent Application Publication
(10) Pub. No.: US 2024/0325350 A1
(43) Pub. Date: Oct. 3, 2024

(54) SPRAYABLE LIQUID SPIRONOLACTONE COMPOSITIONS

(71) Applicant: CELISTA PHARMACEUTICALS LLC, Edina, MN (US)

(72) Inventor: Yogesh DANDIKER, Edina, MN (US)

(21) Appl. No.: 18/619,890

(22) Filed: Mar. 28, 2024

Related U.S. Application Data
(60) Provisional application No. 63/493,211, filed on Mar. 30, 2023.

(51) Int. Cl. A61K 31/365; A61K 9/70; A61K 31/455; A61K 47/10; A61K 47/36

(52) U.S. Cl. CPC A61K 31/365; A61K 9/7015; A61K 31/455; A61K 47/10; A61K 47/36

(57) ABSTRACT

The present disclosure provides a sprayable liquid composition comprising about 1% w/v to about 20% w/v spironolactone or canrenone, about 30% w/v to about 97% w/v aliphatic solvent, about 1% w/v to about 10% w/v water, one or more film forming excipients, wherein the film forming excipient has a solubility in water at a pH between 1 and 10, and a penetration enhancer, wherein the composition forms a washable and/or a peelable film when sprayed on a skin surface, and wherein the spironolactone does not crystallize when the composition is applied to the skin surface. The disclosure also provides a method of treating acne, male and female pattern hair loss, hirsutism, hidradenitis suppurativa, polycystic ovary syndrome (PCOS), or combinations thereof in a subject in need thereof, the method comprising topically applying the composition described herein as a spray to a skin surface of the subject.

### FIELD OF THE INVENTION

The present disclosure provides about 1% w/v to about 20% w/v spironolactone or canrenone, about 30% w/v to about 97% w/v aliphatic solvent, about 1% w/v to about 10% w/v water, one or more film forming excipients, and a penetration enhancer. The disclosure further provides a method of treating acne, male and female pattern hair loss, hirsutism, hidradenitis suppurativa, polycystic ovary syndrome (PCOS), or combinations thereof in a human, the method comprising applying the composition as a spray to the skin.

### BACKGROUND

Spironolactone is an aldosterone receptor antagonist used for the treatment of hypertension, hyperaldosteronism, edema due to various conditions, hirsutism (off-label) and hypokalemia. Several studies report that spironolactone also serves as an effective drug alternative to antibiotics for long-term treatment of acne in females. However, the systemic side effects of spironolactone when administered orally restrict its clinical application. When administered orally, spironolactone rapidly metabolizes in the liver into a number of metabolites including canrenone. Spironolactone and its metabolites can cause diuretic effects, menstrual irregularity, breast tenderness and enlargement, dizziness, headaches, nausea and vomiting in women. In men, oral administration of spironolactone and its metabolites can cause a decrease of libido, impotence and gynecomastia. Because of this, spironolactone is currently prescribed off-label to females, but is not prescribed to males. Additionally, oral administration of the recommended dosage of 50 mg-100 mg spironolactone twice a day for the treatment of acne exposes patients to systemic adverse events for an indication that is local.

In order to bypass the hepatic first pass metabolism of spironolactone and prevent the adverse effects of spironolactone and its metabolites that occur when spironolactone is administered orally, topical administration for the delivery of spironolactone has been attempted. For example, U.S. Pat. No. 4,543,351 reports a spironolactone-containing composition for combating acne in the form of a cream to be applied to human skin. U.S. Pat. No. 8,003,690 describes a topical nanoparticulate spironolactone formulation comprising nanoparticles having a mean diameter in the range of from about 300 nm to about 900 nm. A 5% spironolactone topical lotion and cream have been used to treat grade II acne (Kim, 2012). Sebum secretion was treated with a 5% spironolactone gel (Yamamoto, 1996). However, creams, gels and lotions can be difficult to apply, are inconvenient, and can involve a messy application process and are known for inadvertent transfer of spironolactone to others who may come in contact with these formulations. Further, none of the previous treatment methods have been demonstrated to be effective and safe in treating acne in both men and women.

### SUMMARY OF THE INVENTION

The present disclosure is directed to a composition comprising about 1% w/v to about 20% w/v spironolactone or canrenone, about 30% w/v to about 97% w/v aliphatic solvent, about 1% w/v to about 10% w/v water, one or more film forming excipients, wherein the film forming excipient has a solubility in water at a pH between 1 and 10, and a penetration enhancer, wherein the composition forms a washable and/or a peelable film when sprayed on a skin surface, and wherein the spironolactone does not crystallize when the composition is applied to the skin surface.

In one embodiment, the composition comprises spironolactone. In some embodiments, the composition comprises about 3% w/v to about 10% w/v spironolactone. In some embodiments, the composition comprises about 5% w/v spironolactone.

In some embodiments, the aliphatic solvent is acetone, di-isopropyl adipate, dimethyl isosorbide, dimethyl sulphoxide, ethyl acetate, ethanol, isopropyl alcohol, or combinations thereof. In one embodiment, the aliphatic solvent is ethyl acetate, ethanol, isopropyl alcohol, or combinations thereof. In some embodiments, the aliphatic solvent is a combination of ethyl acetate, ethanol, and isopropyl alcohol.

In one embodiment, the aliphatic solvent is about 15% w/v to about 60% w/v ethyl acetate, about 10% w/v to about 65% w/v di-isopropyl adipate, and about 5% w/v to about 60% w/v ethanol.

In some embodiments, the film forming excipient comprises a polyacrylate polymer, a vinylpyrrolidone polymer, methacrylic acid and methyl methacrylate copolymer 1:1, methacrylic acid and methyl methacrylate copolymer 1:2, poly(butylmethacrylate-co-(2-dimethylaminoethyl)methacrylate-co-methyl methacrylate 1:2:1, hypromellose, hydroxypropyl cellulose, ethyl cellulose, butyl methacrylate and methyl copolymer (3:1), polyvinylpyrrolidone, polyvinylpyrrolidone, polyvinyl acetate or combinations thereof.

In some embodiments, the film forming excipient is butyl methacrylate and methyl copolymer (3:1), polyvinylpyrrolidone or combinations thereof.

In some embodiments, the film forming excipient is about 1% to about 10% w/v of the composition.

In some embodiments, the penetration enhancer is azone, isopropyl myristate, octisalate, oleic acid, Transcutol® P or combinations thereof. In some embodiments, the penetration enhancer is octisalate.

In some embodiments, the penetration enhancer is about 1% to about 10% w/v of the composition.

In some embodiments, the film forming excipient is butyl methacrylate and methyl copolymer (3:1), polyvinylpyrrolidone or combinations thereof, and the penetration enhancer is octisalate.

In some embodiments, the composition further comprises a washability enhancer. In some embodiments, the washability enhancer is polyethylene glycol 400.

In some embodiments, the composition further comprises a buffer. In some embodiments, the buffer comprises sodium citrate, citric acid buffer, or combinations thereof.

In some embodiments, the composition further comprises an additional active agent. In some embodiments, the additional active agent is minoxidil, finasteride, dutasteride, niacinamide or combinations thereof. In some embodiments, the additional active agent is niacinamide.

In some embodiments, the additional active agent is about 1% w/w to about 10% w/w minoxidil, about 0.1% w/w to about 1.0% w/w finasteride or dutasteride, about 1% w/w to about 10% w/w minoxidil and about 0.1% w/w to about 1.0% w/w finasteride or dutasteride, or about 2% w/v to about 10% w/v niacinamide.

In some embodiments, the pH of the composition is about 4 to about 5. In some embodiments, the pH of the composition is about 4.5.

In some embodiments, the composition forms a barrier film less than three minutes after application to the skin surface. In some embodiments, the composition forms a barrier film less than one minute after application to the skin.

In some embodiments, the composition has a viscosity of less than 30 cPs at room temperature. In some embodiments, the composition has a water vapor transmission rate, as a fraction compared to a non-occluded control, not less than 0.50 over a 48-hour period when applied and dried on a porous substrate.

In some embodiments, the present disclosure provides a spray container comprising the composition, and a metering valve. In some embodiments, the spray container further comprises a dose indicator. In some embodiments, the container is pressurized. In some embodiments, the container further comprises a propellent.

In one embodiment, the present disclosure is directed to a film composition comprising spironolactone, octisalate, butyl methacrylate and methyl copolymer (3:1), and polyethylene glycol 400.

In some embodiments, the film composition comprises about 3% w/w to about 70% w/w spironolactone. In some embodiments, the film composition comprises about 20% w/w to about 50% w/w spironolactone.

In some embodiments, the film composition comprises about 3% w/v to about 60% w/v octisalate. In some embodiments, the film composition comprises about 20% w/v to about 60% w/v octisalate.

In some embodiments, the film composition comprises about 3% w/v to about 60% w/v butyl methacrylate and methyl copolymer (3:1). In some embodiments, the film composition comprises about 20% w/v to about 60% w/v methacrylic acid and methyl methacrylate copolymer 1:2.

In some embodiments, the disclosure provides a method of treating acne, male and female pattern hair loss, hirsutism, hidradenitis suppurativa, polycystic ovary syndrome (PCOS), or combinations thereof in a subject, the method comprising topically applying the composition as a spray to a skin surface of the subject. In some embodiments, the composition is applied only once within a 24 hour period.

In some embodiments, the composition is applied at least as a first application at a first timepoint and a second application at a second timepoint, wherein the second timepoint is at least 24 hours after the first timepoint.

In some embodiments, the composition is applied at three or more applications at three or more timepoints. In some embodiments, the time between each timepoint is about 24 hours.

In some embodiments, the first application and the second application are applied at the same site on the skin surface of the subject. In some embodiments, the first application and the second application are applied at different sites on the skin surface of the subject. In some embodiments, the barrier film formed from the first application is removed before applying the second application. In some embodiments, the barrier film formed from the previous application is removed before applying the subsequent application.

In some embodiments, the subject is human. In some embodiments, the subject is female.

In some embodiments, the composition is applied to the skin surface in a single actuation in a volume of about 500 μL or less. In some embodiments, the composition is applied to the skin surface in a single actuation in a volume of about 250 μL or less. In some embodiments, the composition is applied to the skin surface in a single actuation in a volume of about is about 100 μL or less. In some embodiments, the composition is applied to the skin surface in a single actuation in a volume of about 50 μL to about 300 μL.

In one embodiment, the present disclosure is directed to a dual chamber device comprising a first chamber comprising a first composition comprising 1% w/w to 10% w/w spironolactone, about 15% w/w to 20% w/w ethyl acetate and about 10% di-isopropyl adipate, a second chamber comprising a second composition comprising about 50% w/w to about 60% w/w ethanol, about 4% to 7% octisalate, about 5% w/v to about 7% w/v butyl methacrylate and methyl copolymer (3:1) and about 1.5 w/w polyethylene glycol 400, wherein the dual chamber device is suitable for providing a spray comprising the composition.

In some embodiments, the first composition further comprises polyvinylpyrrolidone. In some embodiments, the contents of the first and second chambers are mixed during spraying of the composition onto human skin.

### BRIEF DESCRIPTION OF THE FIGURES

FIG. 1 is a graph illustrating the skin permeation profiles of a 5% and a 3% spironolactone film forming composition as outlined in Example 5.

FIG. 2 is a graph illustrating the results of a film breathability study performed using an occluded and a non-occluded spironolactone film forming composition as outlined in Example 6.

### DETAILED DESCRIPTION OF THE INVENTION

The present disclosure relates to sprayable liquid compositions comprising spironolactone or canrenone and methods suitable for the treatment of acne, male and female pattern hair loss, hirsutism, hidradenitis suppurativa, polycystic ovary syndrome (PCOS), or combinations thereof. In some embodiments, the disclosure provides a sprayable liquid composition comprising spironolactone or canrenone, e.g., a topical spray. Spironolactone compositions have been used previously in the forms of topical lotions, sprays and gels. However, the present disclosure provides for a new composition which is a sprayable liquid, which in some embodiments can form a film on the skin after the spray has dried. The film can act as barrier that prevents transfer of spironolactone and canrenone from the application site to other individuals, e.g., the user's partner or individuals in close proximity. Additionally, the film can be washable as it can conveniently be removed with water while performing daily activities such as a shower. In some embodiments, the sprayable liquid compositions described herein can be sprayed directly on the skin surface, and do not need to be spread over the skin by touching with fingers or with use of a separate applicator. In some embodiments, drug absorption into the skin is rapid. In some embodiments drug absorption from the sprayable liquid composition into the skin is complete when the protective film has dried (<5 mins). In some embodiments, the film can be washed off once it has dried. In some embodiments, during solvent evaporation, the excipients included in the sprayable liquid compositions can prevent recrystallization of spironolactone and canrenone, a feature that can be important for its penetration of the skin. In some embodiments, the spironolactone and canrenone in the sprayable liquid composition described herein do not crystallize when the composition is applied to the skin. In some embodiments, the protective film is breathable which prevents erythema of the skin from developing over long-term use. In some embodiments, direct application to the skin via a sprayable liquid composition provides for increased convenience, and lower transmission of the spironolactone and canrenone to the hands/fingers or other body parts for which it is not needed. In some embodiments, the sprayable liquid compositions described herein can be applied quickly. In some embodiments, the sprayable liquid compositions described herein can be applied quickly over a large area of the skin.

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular. As used herein, “a” or “an” may mean one or more. A used herein, when used in conjunction with the word “comprising,” the words “a” or “an” may mean one or more than one. As used herein, “another” or “a further” may mean at least a second or more.

Throughout this application, the term “about” is used to indicate that a value includes the inherent variation of error for the method/device being employed to determine the value, or the variation that exists among the study subjects. Typically, the term “about” is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability, depending on the situation.

The use of the term “or” in the claims is used to mean “and/or”, unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.,” (whether italicized or not) means that the specific terms recited are representative examples and embodiments of the disclosure that are not intended to be limited to the specific examples referenced or cited unless explicitly stated otherwise.

As used herein, “between” is a range inclusive of the ends of the range. For example, a number between x and y explicitly includes the numbers x and y, and any numbers that fall within x and y.

As used herein, “room temperature” is an indoor temperature suitable for long term storage of biological matter and laboratory experimentation, typically ranging between 15-28° C. In embodiments, room temperature is from 20-25° C.

The present disclosure is directed to a sprayable liquid composition comprising about 1% w/v to about 20% w/v spironolactone or canrenone, about 30% w/v to about 97% w/v aliphatic solvent, about 1% w/v to about 10% w/v water, one or more film forming excipients, wherein the film forming excipient has a solubility in water at a pH between 1 and 10, and a penetration enhancer, wherein the composition forms a washable film and/or a peelable film when sprayed on a skin surface, and wherein the spironolactone does not crystallize when the composition is applied to the skin surface. In some embodiments, the composition comprises spironolactone. In some embodiments, the composition comprises canrenone.

Various concentrations of spironolactone or canrenone can be used in the composition. In some embodiments, the composition comprises about 1% w/v to about 20% w/v spironolactone or canrenone. In some embodiments, the composition comprises about 1% w/v to about 19% w/v spironolactone or canrenone. In some embodiments, the composition comprises about 1% w/v to about 18% w/v spironolactone or canrenone. In some embodiments, the composition comprises about 1% w/v to about 17% w/v spironolactone or canrenone. In some embodiments, the composition comprises about 1% w/v to about 16% w/v spironolactone or canrenone. In some embodiments, the composition comprises about 1% w/v to about 15% w/v spironolactone or canrenone. In some embodiments, the composition comprises about 1% w/v to about 14% w/v spironolactone or canrenone. In some embodiments, the composition comprises about 1% w/v to about 13% w/v spironolactone or canrenone. In some embodiments, the composition comprises about 1% w/v to about 12% w/v spironolactone or canrenone. In some embodiments, the composition comprises about 1% w/v to about 11% w/v spironolactone or canrenone. In some embodiments, the composition comprises about 1% w/v to about 10% w/v spironolactone or canrenone. In some embodiments, the composition comprises about 1% w/v to about 9% w/v spironolactone or canrenone. In some embodiments, the composition comprises about 1% w/v to about 8% w/v spironolactone or canrenone. In some embodiments, the composition comprises about 1% w/v to about 7% w/v spironolactone or canrenone. In some embodiments, the composition comprises about 1% w/v to about 6% w/v spironolactone or canrenone. In some embodiments, the composition comprises about 1% w/v to about 5% w/v spironolactone or canrenone. In some embodiments, the composition comprises about 1% w/v to about 4% w/v spironolactone or canrenone. In some embodiments, the composition comprises about 1% w/v to about 3% w/v spironolactone or canrenone.

In some embodiments, the composition comprises about 2% w/v to about 20% w/v spironolactone or canrenone. In some embodiments, the composition comprises about 2% w/v to about 19% w/v spironolactone or canrenone. In some embodiments, the composition comprises about 2% w/v to about 18% w/v spironolactone or canrenone. In some embodiments, the composition comprises about 2% w/v to about 17% w/v spironolactone or canrenone. In some embodiments, the composition comprises about 2% w/v to about 16% w/v spironolactone or canrenone. In some embodiments, the composition comprises about 2% w/v to about 15% w/v spironolactone or canrenone. In some embodiments, the composition comprises about 2% w/v to about 14% w/v spironolactone or canrenone. In some embodiments, the composition comprises about 2% w/v to about 13% w/v spironolactone or canrenone. In some embodiments, the composition comprises about 2% w/v to about 12% w/v spironolactone or canrenone. In some embodiments, the composition comprises about 2% w/v to about 11% w/v spironolactone or canrenone. In some embodiments, the composition comprises about 2% w/v to about 10% w/v spironolactone or canrenone. In some embodiments, the composition comprises about 2% w/v to about 9% w/v spironolactone or canrenone. In some embodiments, the composition comprises about 2% w/v to about 8% w/v spironolactone or canrenone. In some embodiments, the composition comprises about 2% w/v to about 7% w/v spironolactone or canrenone. In some embodiments, the composition comprises about 2% w/v to about 6% w/v spironolactone or canrenone. In some embodiments, the composition comprises about 2% w/v to about 5% w/v spironolactone or canrenone. In some embodiments, the composition comprises about 2% w/v to about 4% w/v spironolactone or canrenone. In some embodiments, the composition comprises about 2% w/v to about 3% w/v spironolactone or canrenone.

In some embodiments, the composition comprises about 3% w/v to about 20% w/v spironolactone or canrenone. In some embodiments, the composition comprises about 3% w/v to about 19% w/v spironolactone or canrenone. In some embodiments, the composition comprises about 3% w/v to about 18% w/v spironolactone or canrenone. In some embodiments, the composition comprises about 3% w/v to about 17% w/v spironolactone or canrenone. In some embodiments, the composition comprises about 3% w/v to about 16% w/v spironolactone or canrenone. In some embodiments, the composition comprises about 3% w/v to about 15% w/v spironolactone or canrenone. In some embodiments, the composition comprises about 3% w/v to about 14% w/v spironolactone or canrenone. In some embodiments, the composition comprises about 3% w/v to about 13% w/v spironolactone or canrenone. In some embodiments, the composition comprises about 3% w/v to about 12% w/v spironolactone or canrenone. In some embodiments, the composition comprises about 3% w/v to about 11% w/v spironolactone or canrenone. In some embodiments, the composition comprises about 3% w/v to about 10% w/v spironolactone or canrenone. In some embodiments, the composition comprises about 3% w/v to about 9% w/v spironolactone or canrenone. In some embodiments, the composition comprises about 3% w/v to about 8% w/v spironolactone or canrenone. In some embodiments, the composition comprises about 3% w/v to about 7% w/v spironolactone or canrenone. In some embodiments, the composition comprises about 3% w/v to about 6% w/v spironolactone or canrenone. In some embodiments, the composition comprises about 3% w/v to about 5% w/v spironolactone or canrenone. In some embodiments, the composition comprises about 3% w/v to about 4% w/v spironolactone or canrenone.

In some embodiments, the composition comprises about 4% w/v to about 20% w/v spironolactone or canrenone. In some embodiments, the composition comprises about 4% w/v to about 19% w/v spironolactone or canrenone. In some embodiments, the composition comprises about 4% w/v to about 18% w/v spironolactone or canrenone. In some embodiments, the composition comprises about 4% w/v to about 17% w/v spironolactone or canrenone. In some embodiments, the composition comprises about 4% w/v to about 16% w/v spironolactone or canrenone. In some embodiments, the composition comprises about 4% w/v to about 15% w/v spironolactone or canrenone. In some embodiments, the composition comprises about 4% w/v to about 14% w/v spironolactone or canrenone. In some embodiments, the composition comprises about 4% w/v to about 13% w/v spironolactone or canrenone. In some embodiments, the composition comprises about 4% w/v to about 12% w/v spironolactone or canrenone. In some embodiments, the composition comprises about 4% w/v to about 11% w/v spironolactone or canrenone. In some embodiments, the composition comprises about 4% w/v to about 10% w/v spironolactone or canrenone. In some embodiments, the composition comprises about 4% w/v to about 9% w/v spironolactone or canrenone. In some embodiments, the composition comprises about 4% w/v to about 8% w/v spironolactone or canrenone. In some embodiments, the composition comprises about 4% w/v to about 7% w/v spironolactone or canrenone. In some embodiments, the composition comprises about 4% w/v to about 6% w/v spironolactone or canrenone. In some embodiments, the composition comprises about 4% w/v to about 5% w/v spironolactone or canrenone.

In some embodiments, the composition comprises about 5% w/v to about 20% w/v spironolactone or canrenone. In some embodiments, the composition comprises about 5% w/v to about 19% w/v spironolactone or canrenone. In some embodiments, the composition comprises about 5% w/v to about 18% w/v spironolactone or canrenone. In some embodiments, the composition comprises about 5% w/v to about 17% w/v spironolactone or canrenone. In some embodiments, the composition comprises about 5% w/v to about 16% w/v spironolactone or canrenone. In some embodiments, the composition comprises about 5% w/v to about 15% w/v spironolactone or canrenone. In some embodiments, the composition comprises about 5% w/v to about 14% w/v spironolactone or canrenone. In some embodiments, the composition comprises about 5% w/v to about 13% w/v spironolactone or canrenone. In some embodiments, the composition comprises about 5% w/v to about 12% w/v spironolactone or canrenone. In some embodiments, the composition comprises about 5% w/v to about 11% w/v spironolactone or canrenone. In some embodiments, the composition comprises about 5% w/v to about 10% w/v spironolactone or canrenone. In some embodiments, the composition comprises about 5% w/v to about 9% w/v spironolactone or canrenone. In some embodiments, the composition comprises about 5% w/v to about 8% w/v spironolactone or canrenone. In some embodiments, the composition comprises about 5% w/v to about 7% w/v spironolactone or canrenone. In some embodiments, the composition comprises about 5% w/v to about 6% w/v spironolactone or canrenone.

In some embodiments, the composition comprises about 6% w/v to about 20% w/v spironolactone or canrenone. In some embodiments, the composition comprises about 6% w/v to about 19% w/v spironolactone or canrenone. In some embodiments, the composition comprises about 6% w/v to about 18% w/v spironolactone or canrenone. In some embodiments, the composition comprises about 6% w/v to about 17% w/v spironolactone or canrenone. In some embodiments, the composition comprises about 6% w/v to about 16% w/v spironolactone or canrenone. In some embodiments, the composition comprises about 6% w/v to about 15% w/v spironolactone or canrenone. In some embodiments, the composition comprises about 6% w/v to about 14% w/v spironolactone or canrenone. In some embodiments, the composition comprises about 6% w/v to about 13% w/v spironolactone or canrenone. In some embodiments, the composition comprises about 6% w/v to about 12% w/v spironolactone or canrenone. In some embodiments, the composition comprises about 6% w/v to about 11% w/v spironolactone or canrenone. In some embodiments, the composition comprises about 6% w/v to about 10% w/v spironolactone or canrenone. In some embodiments, the composition comprises about 6% w/v to about 9% w/v spironolactone or canrenone. In some embodiments, the composition comprises about 6% w/v to about 8% w/v spironolactone or canrenone. In some embodiments, the composition comprises about 6% w/v to about 7% w/v spironolactone or canrenone.

In some embodiments, the composition comprises about 7% w/v to about 20% w/v spironolactone or canrenone. In some embodiments, the composition comprises about 7% w/v to about 19% w/v spironolactone or canrenone. In some embodiments, the composition comprises about 7% w/v to about 18% w/v spironolactone or canrenone. In some embodiments, the composition comprises about 7% w/v to about 17% w/v spironolactone or canrenone. In some embodiments, the composition comprises about 7% w/v to about 16% w/v spironolactone or canrenone. In some embodiments, the composition comprises about 7% w/v to about 15% w/v spironolactone or canrenone. In some embodiments, the composition comprises about 7% w/v to about 14% w/v spironolactone or canrenone. In some embodiments, the composition comprises about 7% w/v to about 13% w/v spironolactone or canrenone. In some embodiments, the composition comprises about 7% w/v to about 12% w/v spironolactone or canrenone. In some embodiments, the composition comprises about 7% w/v to about 11% w/v spironolactone or canrenone. In some embodiments, the composition comprises about 7% w/v to about 10% w/v spironolactone or canrenone. In some embodiments, the composition comprises about 7% w/v to about 9% w/v spironolactone or canrenone. In some embodiments, the composition comprises about 7% w/v to about 8% w/v spironolactone or canrenone.

In some embodiments, the composition comprises about 8% w/v to about 20% w/v spironolactone or canrenone. In some embodiments, the composition comprises about 8% w/v to about 19% w/v spironolactone or canrenone. In some embodiments, the composition comprises about 8% w/v to about 18% w/v spironolactone or canrenone. In some embodiments, the composition comprises about 8% w/v to about 17% w/v spironolactone or canrenone. In some embodiments, the composition comprises about 8% w/v to about 16% w/v spironolactone or canrenone. In some embodiments, the composition comprises about 8% w/v to about 15% w/v spironolactone or canrenone. In some embodiments, the composition comprises about 8% w/v to about 14% w/v spironolactone or canrenone. In some embodiments, the composition comprises about 8% w/v to about 13% w/v spironolactone or canrenone. In some embodiments, the composition comprises about 8% w/v to about 12% w/v spironolactone or canrenone. In some embodiments, the composition comprises about 8% w/v to about 11% w/v spironolactone or canrenone. In some embodiments, the composition comprises about 8% w/v to about 10% w/v spironolactone or canrenone. In some embodiments, the composition comprises about 8% w/v to about 9% w/v spironolactone or canrenone.

In some embodiments, the composition comprises about 9% w/v to about 20% w/v spironolactone or canrenone. In some embodiments, the composition comprises about 9% w/v to about 19% w/v spironolactone or canrenone. In some embodiments, the composition comprises about 9% w/v to about 18% w/v spironolactone or canrenone. In some embodiments, the composition comprises about 9% w/v to about 17% w/v spironolactone or canrenone. In some embodiments, the composition comprises about 9% w/v to about 16% w/v spironolactone or canrenone. In some embodiments, the composition comprises about 9% w/v to about 15% w/v spironolactone or canrenone. In some embodiments, the composition comprises about 9% w/v to about 14% w/v spironolactone or canrenone. In some embodiments, the composition comprises about 9% w/v to about 13% w/v spironolactone or canrenone. In some embodiments, the composition comprises about 9% w/v to about 12% w/v spironolactone or canrenone. In some embodiments, the composition comprises about 9% w/v to about 11% w/v spironolactone or canrenone. In some embodiments, the composition comprises about 9% w/v to about 10% w/v spironolactone or canrenone.

In some embodiments, the composition comprises about 10% w/v to about 20% w/v spironolactone or canrenone. In some embodiments, the composition comprises about 10% w/v to about 19% w/v spironolactone or canrenone. In some embodiments, the composition comprises about 10% w/v to about 18% w/v spironolactone or canrenone. In some embodiments, the composition comprises about 10% w/v to about 17% w/v spironolactone or canrenone. In some embodiments, the composition comprises about 10% w/v to about 16% w/v spironolactone or canrenone. In some embodiments, the composition comprises about 10% w/v to about 15% w/v spironolactone or canrenone. In some embodiments, the composition comprises about 10% w/v to about 14% w/v spironolactone or canrenone. In some embodiments, the composition comprises about 10% w/v to about 13% w/v spironolactone or canrenone. In some embodiments, the composition comprises about 10% w/v to about 12% w/v spironolactone or canrenone. In some embodiments, the composition comprises about 10% w/v to about 11% w/v spironolactone or canrenone.

In some embodiments, the composition comprises about 1% spironolactone. In some embodiments, the composition comprises about 2% spironolactone. In some embodiments, the composition comprises about 3% spironolactone. In some embodiments, the composition comprises about 4% spironolactone. In some embodiments, the composition comprises about 5% spironolactone. In some embodiments, the composition comprises about 6% spironolactone. In some embodiments, the composition comprises about 7% spironolactone. In some embodiments, the composition comprises about 8% spironolactone. In some embodiments, the composition comprises about 9% spironolactone. In some embodiments, the composition comprises about 10% spironolactone. In some embodiments, the composition comprises about 11% spironolactone. In some embodiments, the composition comprises about 12% spironolactone. In some embodiments, the composition comprises about 13% spironolactone. In some embodiments, the composition comprises about 14% spironolactone. In some embodiments, the composition comprises about 15% spironolactone. In some embodiments, the composition comprises about 16% spironolactone. In some embodiments, the composition comprises about 17% spironolactone. In some embodiments, the composition comprises about 18% spironolactone. In some embodiments, the composition comprises about 19% spironolactone. In some embodiments, the composition comprises about 20% spironolactone.

In some embodiments, the composition comprises about 1% canrenone. In some embodiments, the composition comprises about 2% canrenone. In some embodiments, the composition comprises about 3% canrenone. In some embodiments, the composition comprises about 4% canrenone. In some embodiments, the composition comprises about 5% canrenone. In some embodiments, the composition comprises about 6% canrenone. In some embodiments, the composition comprises about 7% canrenone. In some embodiments, the composition comprises about 8% canrenone. In some embodiments, the composition comprises about 9% canrenone. In some embodiments, the composition comprises about 10% canrenone. In some embodiments, the composition comprises about 11% canrenone. In some embodiments, the composition comprises about 12% canrenone. In some embodiments, the composition comprises about 13% canrenone.

In some embodiments, the composition comprises about 14% canrenone. In some embodiments, the composition comprises about 15% canrenone. In some embodiments, the composition comprises about 16% canrenone. In some embodiments, the composition comprises about 17% canrenone. In some embodiments, the composition comprises about 18% canrenone. In some embodiments, the composition comprises about 19% canrenone. In some embodiments, the composition comprises about 20% canrenone.

Various aliphatic solvents can be used in the sprayable liquid compositions of the present disclosure. The term “aliphatic solvent” enhancer refers to any solvent comprising compounds that are without a ring structure, e.g., without an aromatic ring structure. In some embodiments, the term “aliphatic solvent” refers to mixtures of saturated, long straight-chain, branched-chain, or cyclic paraffins that have low viscosity, e.g., less than 500 cP, less than 100 cP, less than 50 cP, less than 30 cP, less than 10 cP, or less than 5 cP at 20° C., which are suitable for being applied by spraying. In some embodiments, the aliphatic solvent is suitable for solubilizing the film forming excipients. In some embodiments, the aliphatic solvent is suitable for solubilizing spironolactone or canrenone, e.g., a non-aromatic solvent, which is pharmaceutically acceptable. In some embodiments, the aliphatic solvent is suitable for solubilizing both the film forming excipients and spironolactone and/or canrenone. In some embodiments, the solvent is an alcoholic aliphatic solvent. In some embodiments, the aliphatic solvent is ethylene, isooctane, acetylene, propene, propane, squalene, acetone, ethanol, methanol, propanol, butanol, isopropyl alcohol, di-isopropyl adipate, di-methyl sulphoxide, ethyl acetate, and polyethylene. In some embodiments, aliphatic solvents can include more than one solvent, e.g., a mixture of aliphatic solvents. In some embodiments, the solvent is ethanol or a mixture of ethanol with other aliphatic solvents. In some embodiments, the solvent comprises ethyl acetate, ethanol, isopropyl alcohol, or combinations thereof. In some embodiments, the aliphatic solvent is a combination of ethyl acetate, ethanol, and isopropyl alcohol. In some embodiments, the aliphatic solvent has a viscosity suitable for administering via an aerosol spray or a mist. In some embodiments, the aliphatic solvent can be used to solubilize the sprayable liquid compositions described herein, i.e., the sprayable liquid compositions are a solution.

In some embodiments, the aliphatic solvent comprises two solvents at a ratio of about 1:100 to about 100:1. In some embodiments, the aliphatic solvent comprises two solvents at a ratio of about 5:95 to about 95:5. In some embodiments, the aliphatic solvent comprises two solvents at a ratio of about 20:80 to about 80:20. In some embodiments, the aliphatic solvent comprises two solvents at ratio of about 30:70 to about 70:30. In some embodiments, the aliphatic solvent comprises two solvents at a ratio of about 40:60 to about 60:40. In some embodiments, the aliphatic solvent comprises two solvents at a ratio of about 50:50.

In some embodiments, the aliphatic solvent comprises three solvents at a ratio of about 1:1:1 to about 1:1:10. In some embodiments, the aliphatic solvent comprises three solvents at a ratio of about 1:2:2 to about 1:2:4. In some embodiments, the aliphatic solvent comprises three solvents at a ratio of about 1:3:1 to about 1:3:6. In some embodiments, the aliphatic solvent comprises three solvents at a ratio of about 1:4:1 to about 1:4:12.

The total aliphatic solvent concentration can be around 30% to about 97% weight of the composition, but in some embodiments the aliphatic solvent is in a sufficient quantity to dissolve the other excipients and spironolactone or canrenone. In some embodiments, the composition comprises about 65% weight aliphatic solvent. In some embodiments, the composition comprises about 50% to about 90% by weight aliphatic solvent. In some embodiments, the composition comprises about 30% to about 90%, about 40% to about 90%, about 60% to about 90% or about 70% to about 90% by weight aliphatic solvent. In some embodiments, the composition comprises about 52% to about 68%, about 54% to about 66%, about 56% to about 64% or about 58% to about 62% by weight aliphatic solvent. In some embodiments, the composition comprises about 35% to about 45% by weight aliphatic solvent. In some embodiments, the composition comprises about 70% by weight aliphatic solvent. In some embodiments, the composition comprises less than 80%, less than 70%, less than 65%, less than 62% or less than 61% by weight of aliphatic solvent. In some embodiments, the composition comprises about 70% to about 85% alcoholic solvent, about 72% to about 82% aliphatic solvent, or about 74% to about 80% aliphatic solvent by weight. In some embodiments, the composition comprises about 75%, about 76%, about 77%, about 78%, about 79%, about 80%, about 81%, about 82% or about 83% aliphatic solvent by weight. In some embodiments, the reduced aliphatic solvent concentration can result in reduced adverse effect, e.g., inflammation or irritation.

In some embodiments, the aliphatic solvent comprises ethyl acetate, di-isopropyl adipate, and ethanol. In some embodiments, the ethyl acetate, di-isopropyl adipate, and ethanol are in a ratio of about 1:1:1 to about 1:1:10. In some embodiments, the ethyl acetate, di-isopropyl adipate, and ethanol are in a ratio of about 1:2:2 to about 1:2:4. In some embodiments, the ethyl acetate, di-isopropyl adipate, and ethanol are in a ratio of about 1:3:1 to about 1:3:6. In some embodiments, the ethyl acetate, di-isopropyl adipate, and ethanol are in a ratio of about 1:4:1 to about 1:4:12.

In some embodiments, the sprayable liquid composition is about 15% w/v to about 60% w/v ethyl acetate, about 10% w/v to about 65% w/v di-isopropyl adipate, and about 5% w/v to about 60% w/v ethanol. In some embodiments, the sprayable liquid composition is about 25% w/v to about 50% w/v ethyl acetate, about 20% w/v to about 55% w/v di-isopropyl adipate, and about 10% w/v to about 50% w/v ethanol. In some embodiments, the sprayable liquid composition is about 30% w/v to about 40% w/v ethyl acetate, about 30% w/v to about 40% w/v di-isopropyl adipate, and about 30% w/v to about 50% w/v ethanol. In some embodiments, when the liquid sprayable composition is applied to the skin, the solvent is volatile and evaporates (i.e., dries), leaving a trace amount (or less) of the solvent on the skin. In some embodiments, the aliphatic solvent is a volatile solvent. For example, in some embodiments, the solvent evaporates, i.e., 500 μL, 250 μL, or 100 μL vaporizes in less than two minutes, in less than one minute or in less than 30 seconds after the composition is sprayed onto a surface at room temperature and 1 atmospheric pressure.

In some embodiments, the sprayable liquid compositions of the present disclosure comprises water. In some embodiments, the composition comprises about 1% to about 10%, about 1% to about 9%, about 1% to about 8%, about 1% to about 7%, about 1% to about 6%, or about 1% to about 5% v/v water. In some embodiments, the composition comprises about 2% to about 10%, about 2% to about 9%, about 2% to about 8%, about 2% to about 7%, about 2% to about 6%, or about 2% to about 5% v/v water. In some embodiments, the composition comprises about 3% to about 10%, about 3% to about 9%, about 3% to about 8%, about 3% to about 7%, about 3% to about 6%, or about 3% to about 5% v/v water. In some embodiments, the composition comprises about 4% to about 10%, about 4% to about 9%, about 4% to about 8%, about 4% to about 7%, about 4% to about 6%, or about 4% to about 5% v/v water. In some embodiments, the reduced water concentration decreases the drying time of the sprayable liquid compositions described herein.

The present disclosure provides for spironolactone or canrenone sprayable liquid compositions comprising a film forming excipient. In some embodiments, the disclosure provides for compositions, e.g., topical spray compositions comprising a film forming excipient, wherein the film forming excipient comprises a polyacrylate polymer, polyvinyl polymer or a cellulose polymer. In some embodiments, such compositions provide for uniform drug distribution and dose, increased bioavailability, continuous drug release and longer-lasting effect to the treated area, while minimizing transfer of spironolactone or canrenone. In some embodiments, the long-lasting effect of the present compositions allow for a reduced number of total administrations of the spironolactone or canrenone composition. In some embodiments, the long-lasting effect of the present compositions allow for a reduced frequency of administration of the spironolactone or canrenone composition.

The film forming excipient is an excipient, preferably a polymer, that is soluble in aliphatic solvents, preferably in ethanol or a mixture of ethanol with other solvents. The film forming excipient is also soluble in aqueous solutions, preferably water. Although, for film forming excipients having pH dependent solubility, the pH of the aqueous solution must be above or below the specific trigger pH for that excipient to dissolve. For example, some of these film forming excipients dissolve in aqueous solutions only above pH 6.0, only above pH 7.0, or only below pH 5.0. In some embodiments, the film forming excipient has a solubility in water, at a pH between 1 and 10.

In some embodiments, the sprayable liquid composition comprises a film forming excipient comprising a polyacrylate polymer, polyvinyl polymer or a cellulose polymer. Polyacrylate polymers are commercially available and known to those in the art, and can refer to a group of polymers prepared from acrylate monomers. Polyacrylate polymers can include methacrylic acid and methyl methacrylate copolymer 1:1, methacrylic acid and methyl methacrylate copolymer 1:2, and poly(butylmethacrylate-co-(2-dimethylaminoethyl)methacrylate-co-methyl methacrylate 1:2:1. In some embodiments, the film forming excipient is butyl methacrylate and methyl copolymer (3:1), polyvinylpyrrolidone or combinations thereof. The term cellulose polymer refers to a group of polymers prepared from glucose monomers. Polyvinyl polymers are commercially available and known to those in the art, and can refer to a group of polymers prepared from vinyl monomers. In some embodiments, the film forming excipients used herein can exhibit properties that are important for topical compositions including high tensile strength, lack of deformity under minimal tensile stress, and low sensitivity to microbial contamination. In some embodiments, the film forming excipient allows for penetration of spironolactone or canrenone into the skin. In some embodiments, the film forming excipient allows moisture vapor to pass through the skin.

In some embodiments, the film forming excipient is a polyacrylate polymer. In some embodiments, the polyacrylate polymer is methacrylic acid and methyl methacrylate copolymer. In some embodiments, the methacrylic acid and methacrylate copolymer are in a ratio of about 1:4 to about 4:1, about 1:3 to about 3:1, about 1:2 to about 2:1. In some embodiments, the methacrylic acid and methacrylate copolymer are in ratio of 1:1. In some embodiments, the methacrylic acid and methacrylate copolymer are in ratio of 1:2. In some embodiments the polyacrylate polymer is methacrylic acid and methyl methacrylate copolymer 1:2. In some embodiments the polyacrylate polymer is poly(butylmethacrylate-co-(2-dimethylaminoethyl)methacrylate-co-methyl methacrylate 1:2:1. Polyvinyl polymers can include polyvinyl alcohol, polyvinylpyrrolidone, and polyvinyl acetate. Various cellulose polymers are known in the art, and can include hypromellose, hydroxypropyl cellulose, and ethyl cellulose.

In some embodiments, the cellulose polymer is hypromellose. In some embodiments the cellulose polymer is hydroxypropyl cellulose. In some embodiments the cellulose polymer is ethyl cellulose. In some embodiments, the sprayable liquid compositions can have various combinations of penetration enhancers and film forming excipients. In some embodiments, the composition comprises the penetration enhancer octisalate, and the film forming excipient methacrylic acid and methyl methacrylate copolymer 1:1.

In some embodiments, the sprayable liquid composition can have more than one film forming excipient. In some embodiments, the composition comprises about 1% w/v to about 10% w/v film forming excipient. In some embodiments, the composition comprises about 1% w/v to about 9% w/v film forming excipient. In some embodiments, the composition comprises about 1% w/v to about 8% w/v film forming excipient. In some embodiments, the composition comprises about 1% w/v to about 7% w/v film forming excipient. In some embodiments, the composition comprises about 1% w/v to about 6% w/v film forming excipient. In some embodiments, the composition comprises about 1% w/v to about 5% w/v film forming excipient. In some embodiments, the composition comprises about 1% w/v to about 4% w/v film forming excipient. In some embodiments, the composition comprises about 1% w/v to about 3% w/v film forming excipient. In some embodiments, the composition comprises about 1% w/v to about 2% w/v film forming excipient.

In some embodiments, the sprayable liquid composition comprises about 2% w/v to about 10% w/v film forming excipient. In some embodiments, the composition comprises about 2% w/v to about 9% w/v film forming excipient. In some embodiments, the composition comprises about 2% w/v to about 8% w/v film forming excipient. In some embodiments, the composition comprises about 2% w/v to about 7% w/v film forming excipient. In some embodiments, the composition comprises about 2% w/v to about 6% w/v film forming excipient. In some embodiments, the composition comprises about 2% w/v to about 5% w/v film forming excipient. In some embodiments, the composition comprises about 2% w/v to about 4% w/v film forming excipient. In some embodiments, the composition comprises about 2% w/v to about 3% w/v film forming excipient.

In some embodiments, the sprayable liquid composition comprises about 3% w/v to about 10% w/v film forming excipient. In some embodiments, the composition comprises about 3% w/v to about 9% w/v film forming excipient. In some embodiments, the composition comprises about 3% w/v to about 8% w/v film forming excipient. In some embodiments, the composition comprises about 3% w/v to about 7% w/v film forming excipient. In some embodiments, the composition comprises about 3% w/v to about 6% w/v film forming excipient. In some embodiments, the composition comprises about 3% w/v to about 5% w/v film forming excipient. In some embodiments, the composition comprises about 3% w/v to about 4% w/v film forming excipient.

In some embodiments, the sprayable liquid composition comprises about 4% w/v to about 10% w/v film forming excipient. In some embodiments, the composition comprises about 4% w/v to about 9% w/v film forming excipient. In some embodiments, the composition comprises about 4% w/v to about 8% w/v film forming excipient. In some embodiments, the composition comprises about 4% w/v to about 7% w/v film forming excipient. In some embodiments, the composition comprises about 4% w/v to about 6% w/v film forming excipient. In some embodiments, the composition comprises about 4% w/v to about 5% w/v film forming excipient.

In some embodiments, the sprayable liquid composition comprises about 5% w/v to about 10% w/v film forming excipient. In some embodiments, the composition comprises about 5% w/v to about 9% w/v film forming excipient. In some embodiments, the composition comprises about 5% w/v to about 8% w/v film forming excipient. In some embodiments, the composition comprises about 5% w/v to about 7% w/v film forming excipient. In some embodiments, the composition comprises about 5% w/v to about 6% w/v film forming excipient.

In some embodiments, the sprayable liquid composition comprises about 6% w/v to about 10% w/v film forming excipient. In some embodiments, the composition comprises about 6% w/v to about 9% w/v film forming excipient. In some embodiments, the composition comprises about 6% w/v to about 8% w/v film forming excipient. In some embodiments, the composition comprises about 6% w/v to about 7% w/v film forming excipient. In some embodiments, the composition comprises about 7% w/v to about 10% w/v film forming excipient. In some embodiments, the composition comprises about 7% w/v to about 9% w/v film forming excipient.

In some embodiments, the composition comprises about 7% w/v to about 8% w/v film forming excipient. In some embodiments, the composition comprises about 8% w/v to about 10% w/v film forming excipient. In some embodiments, the composition comprises about 8% w/v to about 9% w/v film forming excipient. In some embodiments, the composition comprises about 9% w/v to about 10% w/v film forming excipient.

In some embodiments, the composition comprises about 5% w/v to about 7% w/v film forming excipient. In some embodiments, the composition comprises about 5% w/v to about 7% w/v film forming excipient. In some embodiments, the composition comprises about 5% w/v to about 7% w/v film forming excipient. In some embodiments, the composition comprises about 6% w/v to about 7% w/v film forming excipient.

In some embodiments, the sprayable liquid composition can comprise a penetration enhancer. The term “penetration enhancer” refers to any compound or composition that penetrate into the skin and interact with skin constituents to promote drug flux or reversibly decrease the barrier resistance. Penetration enhancers can include substances which promote the dermal absorption of spironolactone or canrenone through the skin temporarily by transiently disturbing the lipid bilayer structure in the stratum corneum barrier or enhancing the solubility of the drug in the skin to facilitate drug delivery. In some embodiments, the penetration enhancer can include any penetration enhancer known to be pharmaceutically acceptable. In some embodiments, the penetration enhancer can include any skin enhancer as found in the U.S. Pharmacopeia, or otherwise known in the art, e.g., M. E. Lane, Skin Permeation Enhancers, Int. J. Pharma 447(1-2):12-21 (2013). In some embodiments, the penetration enhancer can include 1-dodecylazacycloheptan-2-one, isopropyl myristate, octisalate, oleic acid, diethylene glycol monoethyl ether (Transcutol®), or combination thereof. In some embodiments, one, two, three or greater than three penetration enhancers are on the sprayable liquid composition. In some embodiments, two penetration enhancers are in the composition. In some embodiments, the penetration enhancer is azone, isopropyl myristate, octisalate, oleic acid, Transcutol® P or combinations thereof. In some embodiments, the penetration enhancer is octisalate.

Various concentrations of penetration enhancers can be present in the sprayable liquid composition. The permeation enhancer can be adjusted to facilitate the penetration of spironolactone or canrenone. In some embodiments, the composition comprises about 0.1% w/v to about 20% w/v, from about 0.1% w/v to about 15% w/v, from about 0.1% w/v to about 10% w/v, about 0.1% w/v to about 5% w/v, or about 0.5% w/v to about 8% w/v penetration enhancer. In some embodiments, the composition comprises about 4% to about 25% by weight penetration enhancer. In some embodiments, the composition comprises about 4% to about 20% by weight penetration enhancer. In some embodiments, the composition comprises about 4% to about 15% by weight penetration enhancer. In some embodiments, the composition comprises about 4% to about 10% by weight penetration enhancer. In some embodiments, the composition comprises about 10% to about 15% by weight penetration enhancer. In some embodiments, the composition comprises about 10% to about 20% by weight penetration enhancer. In some embodiments, the composition comprises about 15% to about 20% by weight penetration enhancer. In some embodiments, the composition comprises about 15% to about 25% by weight penetration enhancer.

In some embodiments, the sprayable liquid composition comprises about 1% to about 10% of the penetration enhancer. In some embodiments, the composition comprises about 2% to about 10% of the penetration enhancer. In some embodiments, the composition comprises about 3% to about 10% of the penetration enhancer. In some embodiments, the composition comprises about 4% to about 10% of the penetration enhancer. In some embodiments, the composition comprises about 5% to about 10% of the penetration enhancer. In some embodiments, the composition comprises about 6% to about 10% of the penetration enhancer. In some embodiments, the composition comprises about 7% to about 10% of the penetration enhancer. In some embodiments, the composition comprises about 8% to about 10% of the penetration enhancer. In some embodiments, the composition comprises about 9% to about 10% of the penetration enhancer.

In some embodiments, the film forming excipient is butyl methacrylate and methyl copolymer (3:1), polyvinylpyrrolidone or combinations thereof, and the penetration enhancer is octisalate.

In some embodiments, the sprayable liquid compositions comprising the film forming excipients herein form a non-tacky, flexible, adhesive and peelable film, or otherwise removable solid that is coherent as a thin solid layer when dried. As used herein, the term “peelable” refers to materials that can be removably applied to and adhere to surfaces such as the surface of mammalian and human skin and can be subsequently removed from the skin by physical force. Such peelable materials can be effective to exfoliate skin and remove comedones by mechanical means. Peelable films according to the compositions and methods of this invention can be adhesively and removably applied to human skin and, by virtue of being forcibly removed, carry away the dead skin cells, hair, dirt, sebum and blackheads which have adhered to the films while leaving behind the skin benefit agents and active ingredients on the skin. In some embodiments, the peelable films of this invention have an adhesive force of at least about 5 to about 25 ounces (oz). In some embodiments, the peelable films of this invention do not have an adhesive force that cause disruption to the outer layer of the skin and thereby damage the skin. In some embodiments, the adhesive force of the peelable films of this invention are sufficiently high to enable the films to exfoliate and remove comedones from the skin. In some embodiments, the cohesive film can be peelable from the skin, e.g., the cohesive film can remain as a single large piece when peeled from the skin, or tears into two or three large pieces. In some embodiments, the peelable film removes undesirable materials from the skin, for example excessive dead skin cells and comedones.

In some embodiments, the sprayable liquid composition forms a washable film. In some embodiments, the present disclosure provides sprayable liquid compositions that can be washed off via convenient methods, i.e., showering, when absorption of the drug into the skin is complete. In some embodiments, washability includes removal of the film without the use of a detergent. The ability to easily wash off the dried film in the composition allows the user to reapply the subsequent dose of the composition at the previous site of application. A washability enhancer can be included in the composition to make the film more washable, i.e., easier to remove from skin with water or soap and water, particularly when the film forming excipient is an excipient that requires a certain pH to dissolve in water or an aqueous environment, such as methacrylic acid-methyl methacrylate copolymer (1:2) (Eudragit® S100). In some cases, the washability enhancer also acts as a plasticizer. Moreover, the film forming excipient, or mixture of film forming excipient with a plasticizer and/or washability enhancer, prevents recrystallization of spironolactone and canrenone and maintains spironolactone and canrenone in an amorphous state during and after solvent evaporation. In some embodiments, the washability enhancer is a low molecular weight polyethylene glycol (PEG) containing ethylene glycol polymer of various molecular weights, such as polyethylene glycol 300, 400 or 600.

The concentration of washability enhancer in the sprayable liquid composition can be about 1% to about 300% of the weight of the film forming excipient in the composition. In some embodiments it is about 50% to about 250% of the weight of the film forming excipient in the composition. In other embodiments, it is about 20% to about 100% of the weight of the film forming excipient in the composition. It can also be is about 40%, about 45%, about 50%, about 55%, about 60%, about 70%, about 80%, about 90%, about 100%, about 120%, about 140%, about 160%, about 180%, about 190%, about 200%, about 210%, about 220%, about 230%, about 240%, or about 250% of the weight of the film forming excipient in the composition. With regard to the total weight of the composition, in some embodiments, the concentration of washability enhancer in the composition can be about 0.1% w/v to about 20% w/v, about 0.1% w/v to about 15% w/v, about 0.1% w/v to about 10% w/v, about 0.1% w/v to about 5% w/v, about 0.5% w/v to about 5% w/v, or about 1% w/v to about 5% w/v of the composition.

The sprayable liquid composition can include one or more plasticizers such as dibutyl sebacate, triethyl citrate, triacetin, glycerol, a low molecular weight polyethylene glycol, e.g., polyethylene glycol 300, 400 or 600, and/or propylene glycol. The concentration of plasticizer in the composition can be about 0.1% w/v to about 20% w/v, about 0.1% w/v to about 15% w/v, about 0.1% w/v to about 10% w/v, about 0.1% w/v to about 5% w/v, about 0.5% w/v to about 5% w/v, or about 1% w/v to about 5% w/v of the composition. In some embodiments it is about 0.5% w/v, about 1% w/v, about 1.5% w/v, about 2% w/v, about 2.5% w/v, about 3% w/v, about 3.5% w/v, or about 4% w/v, of the composition. In some embodiments, the plasticizer is also a washability enhancer.

The sprayable liquid composition can also include one or more viscosity increasing agents, e.g., povidone, glycerin, hydroxypropyl cellulose, methylcellulose, and/or carboxymethylcellulose. The concentration of the viscosity increasing agent, if included, can be from about 0.1% w/v to about 10% w/v, about 0.1% w/v to about 8% w/v, about 0.1% w/v to about 5% w/v of the composition, e.g., about 0.5% w/v, about 1% w/v, about 1.5% w/v, about 2% w/v, or about 2.5% w/v of the composition. In some embodiments, the viscosity increasing agent is in an amount that allows the composition to still be a sprayable liquid solution. For example, in some embodiments, the viscosity of the composition is less than 40 mPas, less than 30 mPas, less than 20 mPas, less than 10 mPas or less than 5 mPas at room temperature and 1 atmospheric pressure.

In some embodiments, the sprayable liquid compositions can comprise viscosity increasing agents. In some embodiments, the viscosity increasing agent is a bioadhesive. Such agents include Carbopol®. Carbopol® polymers are high molecular weight, crosslinked polyacrylic acid polymers. The polymers differ by crosslink density and can be grouped into the following categories: (1) Carbopol® homopolymers, such as acrylic acid crosslinked with allyl sucrose or allyl pentaerythritol, e.g., Carbopol® 71G NF (viscosity 4,000-11,000), 971P NF (viscosity 4,000-11,000), 974P NF, (viscosity 29,400-39,400), 980 NF (viscosity 40,000-60,000), 981 NF (viscosity 4000-10,000), 5984 EP (viscosity 30,500-39,400), 934 NF (viscosity 30,500-39,400), 934P NF (viscosity 29,400-39,400), 940 NF (viscosity 40,000-60,000), 941 NF (viscosity 4,000-10,000); and (2) Carbopol® copolymers: acrylic acid and C10-C30 alkyl acrylate crosslinked with allyl pentaerythritol, e.g., Carbopol® 1342 NF, viscosity 9,500-26,500.

The compositions of the present disclosure can also include other excipients, added, e.g., to achieve a desired consistency or appearance, or to protect the composition components from degradation and oxidation. Such excipients include, for example, cosolvents, stabilizing agents, antioxidants, humectants, preservatives, pH modifiers, colorants, dyes, and fragrances known in the art of formulation.

Another optional excipient of the compositions is a pH modifier, such as a buffer. In some embodiments, the buffer comprises sodium citrate, citric acid, fumaric acid, monopotassium phosphate, boric acid, acetic acid, adipic acid, calcium hydroxide, glycine, hydrochloric acid, lactic acid, magnesium aluminometasilicates, phosphoric acid, sodium carbonate, sodium hydroxide, sorbic acid, succinic acid, tartaric acid, and derivatives, salts, diethyl barbituric acid or combinations thereof. In some embodiments, the buffer is present in the composition at a concentration of is present in the composition at a concentration from 1 mM to 100 mM, about 1 mM to 50 mM, about 5 mM to 25 mM or about 10 mM. In some embodiments, the buffer is present in the composition at a concentration of 100 mM. In some embodiments, the pH of the sprayable liquid composition is about 3 to about 6, about 3.5 to about 5.5, or about 4 to about 5. In some embodiments, the pH of the sprayable liquid composition is about 4.5.

In some embodiments, the addition of emollients, emulsion stabilizers, moisturizers, excipients, and other compounds may be modified to enhance the sensory properties of the sprayable topical compositions, including but not limited to the skin feel (silkiness, lightness, creaminess, etc.), soften, soothe and moisturize the skin, support renewal of post-procedure skin, help restore the skin's moisture balance, hydrate dry and compromised skin, and is suitable for extremely dry skin. In some embodiments, the composition comprises a moisturizer. In some embodiments, the moisturizer is petrolatum, microcrystalline wax, physalis angulata extract, caprylic/capric triglyceride, butyrospermum parki (shea butter) extract, bisabolol, and tocopherol.

In some embodiments, the composition comprises an additional active agent. In some embodiments, the additional active agent is a vitamin, medication for new hair growth, or an antioxidant. In some embodiments, the additional active agent is niacinamide, minoxidil, finasteride, dutasteride or combinations thereof. In some embodiments, the additional active agent is niacinamide. In some embodiments, the additional active agent is (i) about 1% w/w to about 10% w/w minoxidil, (ii) about 0.1% w/w to about 1.0% w/w finasteride or about 0.1% w/w to about 1.0% w/w dutasteride, (iii) about 1% w/w to about 10% w/w minoxidil and about 0.1% w/w to about 1.0% w/w finasteride or about 0.1% w/w to about 1.0% w/w dutasteride, or (iv) about 2% w/v to about 10% w/v niacinamide. Exemplary compositions comprising the additional agent can comprise the additional active agent in the following weight percentages in the compositions:

| | | | |
|---|---|---|---|
| minoxidil | 1% to 10% | 2% to 8% | 4% to 6% |
| finasteride | 0.1% to 1% | 0.2% to 0.8% | 0.4% to 0.6% |
| dutasteride | 0.1% to 1% | 0.2% to 0.8% | 0.4% to 0.6% |
| niacinamide | 2% to 10% | 3% to 8% | 4% to 6% |

In some embodiments, particularly for application to the skin, the composition can comprise a fragrance or perfume to impart a desired aroma, or to mask odors that can be associated with other components of the composition. In some embodiments, the concentration of the fragrance is about 0.01% w/v to about 5% w/v of the composition, or about 0.1% w/v to about 1% w/v of the composition.

Any fragrance suitable for application to the skin can be used herein including a wide variety of fragrances and perfumes that are known to those skilled in the art. In some embodiments, the amount of fragrance can be effective for providing a noticeable aroma to the composition, or for masking undesired aroma of the composition. In some embodiments, the fragrance does not impart excessive stinging to the skin, especially to broken or irritated skin.

Typical fragrances are described in Arctander, Perfume and Flavour Chemicals (Aroma Chemicals), Vol. I and II (1969, Allured Publishing Corporation, 1969 (ISBN 0931710375, 9780931710377), and Arctander, Perfume and Flavor Materials of Natural Origin (1994, by Allured Pub Corp) (ISBN 0931710367; ISBN13: 9780931710360). Fragrance used in the present composition can also contain solubilizers, diluents, or solvents which are well known in the art.

In some embodiments, application of spironolactone or canrenone compositions without a protective cover or barrier over the area can leave unabsorbed drug exposed, which creates a risk of transfer of spironolactone or canrenone to other persons or surfaces and unwanted side effects. Additionally, in some embodiments, the loss of spironolactone or canrenone after transfer from the user's skin to other surfaces can also alter the amount of the drug remaining on the skin necessary for its intended therapeutic effect. The present disclosure provides a sprayable liquid composition that prevents transfer of spironolactone or canrenone.

In some embodiments, the barrier film is occlusive. In some embodiments, the occlusive film is transparent. In some embodiments, occlusive refers to a film that forms a protective layer on a surface, meaning the film prevents transference of spironolactone or canrenone to other humans. In some embodiments, the occlusive film forms a protective layer against bacteria and viruses. In some embodiments, the occlusive film is breathable and allows moisture vapor to be able to pass through the film, which maintains the integrity of the skin.

As the barrier film prevents transfer of spironolactone or canrenone, it is important for the sprayable liquid composition to quickly dry after application and form the barrier film. In some embodiments, the sprayable liquid composition dries quickly after application. For example, in some embodiments, the sprayable liquid composition dries within 90 seconds, within 80 seconds, within 70 seconds or within 60 seconds after application. In some embodiments, the sprayable liquid composition forms a barrier film less than three minutes after application to the skin. In some embodiments, the sprayable liquid composition forms a barrier film less than two minutes after application to the skin. In some embodiments, the sprayable liquid composition forms a barrier film in less than one minute after application to the skin. For example, in some embodiments, the sprayable liquid composition forms a barrier film within 90 seconds, within 80 seconds, within 70 seconds or within 60 seconds after application.

In some embodiments, the sprayable liquid composition dries quickly after application. For example, in some embodiments, the sprayable liquid composition dries within 90 seconds, within 80 seconds, within 70 seconds or within 60 seconds after application of 500 μL, 250 μL, 100 μL or 50 μL of the composition to the skin at room temperature and 1 atmospheric pressure. In some embodiments, the sprayable liquid composition forms a barrier film less than three minutes after application of 500 μL, 250 μL, 100 μL or 50 μL of the composition to the skin at room temperature and 1 atmospheric pressure. In some embodiments, the sprayable liquid composition forms a barrier film less than two minutes after application of 500 μL, 250 μL, 100 L or 50 μL of the composition to the skin at room temperature and 1 atmospheric pressure. In some embodiments, the sprayable liquid composition forms a barrier film in less than one minute after application of 500 μL, 250 μL, 100 μL or 50 μL of the composition to the skin at room temperature and 1 atmospheric pressure. For example, in some embodiments, the sprayable liquid composition forms a barrier film within 90 seconds, within 80 seconds, within 70 seconds or within 60 seconds after application of 500 μL, 250 μL, 100 μL or 50 μL of the composition at room temperature and 1 atmospheric pressure.

In some embodiments, the use of transdermal patches or topical solutions with a protective cover or barrier over the area can result in skin sensitization and/or irritation, discomfort from adhesives, and imperfect skin adhesion. The present disclosure provides compositions that are designed to remain intact for an extended period of time, e.g., for 24 hours without the need to cover or otherwise protect the composition. In some embodiments, it is important for moisture vapor to be able to pass through the film during the time the film is present in order to ensure that the underlying skin is able to breathe and allow sweat to evaporate rather than block skin pores. In some embodiments, the present disclosure provides compositions that have a water vapor transmission rate, as a fraction compared to a non-occluded control, not less than 0.50 over a 48 hour period, not less than 1.0 over a 48 hour period, not less than 1.2 over a 48 hour period the composition, when dried on a porous substrate. In some embodiments, the barrier film of the composition prevents reactions of the skin that are associated with non-breathable topical and transdermal treatments such as intense itching, redness (erythema), and blistering.

High viscosity topical spironolactone or canrenone formulations, e.g., creams, lotions and gels, require application to the skin with the palm or fingers of the hand of the user, and thus these types of formulations are associated with a significant risk for secondary spironolactone or canrenone exposure. The present claims feature a “sprayable liquid composition” which refer to liquid, i.e., low viscosity compositions, that can be applied directly to the skin in an amount effective via a spray. Such sprayable liquid composition are useful and convenient in delivering the required dosage or spironolactone or canrenone needed for the treatment of conditions such as acne, male and female pattern hair loss, hirsutism, hidradenitis suppurativa and/or polycystic ovary syndrome (PCOS). In some embodiments, the “sprayable liquid composition” has low viscosity (about 15 cPs to about 45 cPs) and can be administered in smaller volumes, i.e., in a volume less than 500 μL per actuation. In some embodiments, the sprayable liquid composition has a viscosity suitable for administering via an aerosol spray and/or a mist. For example, in some embodiments, the sprayable liquid composition has a viscosity of less than 200 cPs, less than 100 cPs, less than 50 cPs or less than 30 cPs at room temperature. In some embodiments, the sprayable liquid formation has a viscosity of about 1 cp to about 10 cPs, about 5 cPS to about 20 cPs or about 20 cPs to about 30 cPs at room temperature. In some embodiments, the sprayable liquid composition has a viscosity of about 5 cPs to about 15 cPs. As used throughout the present disclosure, viscosity is measured at standard conditions, e.g., 1 atm pressure and 25° C. temperature.

The sprayable liquid composition of the present disclosure can be administered by means to known in the art. In some embodiments, the disclosure provides a spray container for administering the composition. In some embodiments, the disclosure provides a spray container comprising the sprayable liquid compositions as described herein and a metering valve. In some embodiments, the spray container is a sealed and pressurized device, e.g., an aerosol container. In some embodiments, the spray container is a sealed and pressurized device, an aerosol container, with a means for providing an aerosol spray of the sprayable liquid composition, e.g., an aerosol nozzle. In some embodiments, the spray container further comprises a dose indicator. In some embodiments, the spray container comprises a propellant. In some embodiments, the spray container comprises an amount of the sprayable liquid composition sufficient for a single administration of the composition. In some embodiments, the spray container comprises an amount of the sprayable liquid composition sufficient for two administrations of the composition. In some embodiments, the spray container comprises an amount of the sprayable liquid composition sufficient for three or more administrations of the composition.

In some embodiments, the invention comprises a device that compartmentalizes the ingredients until time of application. For example, in one embodiment, one or more ingredients are separated from other ingredients until a user combines them. Such separate delivery helps to ensure that the two fluids are not mixed prematurely, and to ensure that minimal mixed composition is stored within the dispensing device, to ensure high efficacy. In some embodiments, a dual chamber or multi-chamber device is used to hold and/or apply the sprayable liquid composition. In some embodiments, the present disclosure provides a dual chamber device used to apply the compositions described herein. In some embodiments, the dual device chamber comprises a first and second chamber. In some embodiments, the first and second chamber each comprise different compositions. In some embodiments, the compositions in each chamber are kept separate until mixing during application either by concomitant actuation of the sprays from each of the chambers or by application from a pad when a seal is broken between pouches containing each formulation. Separate dip tubes may be provided to pull liquid from the separate chambers, each with its own separate pump. Each pump may deliver the separate compositions to a nozzle (e.g., a spray nozzle) of the device through separate delivery tubes (e.g., from the pumps to the nozzle). Such separate delivery tubes may be preferred for limiting mixing of the components before use. In another embodiment, a single delivery tube from the two pumps may be provided, e.g., where the volume of mixed composition that may be present within such a delivery tube may be sufficiently small so as to still provide overall desired efficacy characteristics.

In some embodiments, the first chamber comprises a first composition comprising 1% w/w to 10% w/w spironolactone, about 15% w/w to 20% w/w ethyl acetate and about 10% di-isopropyl adipate. In some embodiments, the second chamber comprises a second composition comprising about 50% w/w to about 60% w/w ethanol, about 4% to 7% octisalate, about 5% w/v to about 7% w/v butyl methacrylate and methyl copolymer (3:1) and about 1.5 w/w polyethylene glycol 400. In some embodiments, the dual chamber device comprises a first chamber comprising a first composition comprising 1% w/w to 10% w/w spironolactone, about 15% w/w to 20% w/w ethyl acetate and about 10% di-isopropyl adipate, and a second chamber comprising a second composition comprising about 50% w/w to about 60% w/w ethanol, about 4% to 7% octisalate, about 5% w/v to about 7% w/v butyl methacrylate and methyl copolymer (3:1) and about 1.5 w/w polyethylene glycol 400, wherein the dual chamber device is suitable for providing a spray comprising the composition. In some embodiments, the first composition further comprises polyvinylpyrrolidone. In some embodiments, the contents of the first and second chambers are mixed during spraying of the composition onto human skin.

Particular embodiments of the present disclosure may include the administration of one or more additional active agents with the administration of spironolactone and/or canrenone as described herein. The identity of the additional active agent(s) will be largely dependent on the nature of the underlying condition being treated (e.g., the addition of a moisturizer such as niacinamide may be appropriate in the treatment of skin dryness and the addition of agents such as finasteride and dutasteride may be appropriate in the treatment of hair loss in males and females). In some embodiments, the compositions of the device may include additional active agents that are effective in promoting hair growth or preventing hair loss in the treatment of male pattern baldness and/or ameliorating dry skin, rashes, facial flushing, raised red bumps, skin and skin sensitivity in the treatment of skin disorders.

Suitable additional active agents include, but are not limited to, minoxidil, minoxidil sulfate, or another salt form such as chloride, carbonate, nitrate, etc., finasteride, and niacinamide. In some embodiments, the additional active agent is minoxidil, finasteride, dutasteride, niacinamide or combinations thereof. In some embodiments, the additional active agent is minoxidil.

In some embodiments, the additional active agent is about 0.1% to about 10% of the spayable liquid composition. In some embodiments, the additional active agent is about 1% w/w to about 10% w/w minoxidil, about 0.1% w/w to about 1.0% w/w finasteride or dutasteride, about 2% w/v to about 10% w/v niacinamide, or any combination thereof. In some embodiments, additional active agent is present in the same sprayable liquid composition as the spirolactone or canrenone. For example, the additional active agent, e.g., minoxidil can be in the same composition as spirolactone, and the composition can be the same chamber of the dispensing system. In some embodiments, the additional active agent is in a sprayable liquid composition that is distinct from the spayable liquid composition comprising the spirolactone. For example, the additional active agent, e.g., minoxidil, can be in a first composition in first chamber of a dispensing system, and the spirolactone can be in a second composition in a second chamber in the dispensing system. Thus, it is envisioned by the present disclosure that the both the spirolactone and the additional active agent can be applied from the same dispensing system independently of whether the spirolactone and the additional active agent are in the same sprayable liquid composition or in distinct sprayable liquid compositions.

Various containers can be used to hold, store or house the compositions disclosed herein in the spray container or dual chamber device. In some embodiments, the container or device can comprise a metal body, preferably lined with a chemically inert coating material to avoid degradation of the composition due to any interaction between the body and the composition. In some embodiments, a container or device can comprise a plastic body, preferably lined with a chemically inert coating material to avoid degradation of the composition due to any interaction between the body and the composition. In some embodiments, the container or device is a substantially rigid metal or plastic container adapted to contain a pressurized propellant located within the container and in contact with the product to be dispensed. In some embodiments, the container or device is an inner substantially rigid metal or plastic container adapted to contain a pressurized propellant located within an outer container made from the same material and away from contact with the product to be dispensed.

In some embodiments, the container or device body can be constructed from materials such as metal, glass, ceramics, polyester, polyethylene terephthalate (PET) or other polymers. In some embodiments, glass containers can be provided with a safety coating of, for instance, polypropylene to contain glass shards that may be formed on impact with a hard surface. In some embodiments, metal container bodies can be used to withstand impact and are amenable to surface coating. In some embodiments, the container comprises stainless steel, tinplate and aluminum, or combinations thereof. In some embodiments the aluminum is aluminum alloy or anodized aluminum.

Various inert coating materials can be used to line the container body including any suitable coating material known in the art such as a polymer, lacquer, resin or other coating treatment that creates a barrier between the container or device and the composition for preventing any chemical interaction between the composition and the container or device. In some embodiments, the inert material is a non-metallic coating. In some embodiments, known coatings for metal containers include acrylic, phenolic, polyester, epoxy and vinyl resins can be used to line the container body of the drug-device combination disclosed herein. Accordingly, the container or device coating for use with a composition of the present invention can be selected so that it exhibits no acidic or alkaline reactivity in itself, and that no acidic or alkaline reacting impurities are leached from it in the presence of the composition.

In some embodiments, the interior of a metal container or device can be lined with materials such as polyamides, polyimides, polypropylene, polyethylene, fluoropolymers, including perfluoroethylenepropylene copolymer (FEP), fluororubber (FPM), ethylene-propylene diene monomer rubber (EPDM), polytetrafluoroethylene (PTFE), ethylene tetrafluoroethylene copolymer (EFTE), perfluoroalkoxyalkanes, perfluoroalkoxyalkylenes, or blends of fluoropolymers with non-fluorocarbon polymers. Fluoropolymers can, for example, be used in combination with polyimide-polyamide resins.

The coating material of the container or device can be applied as a single layer, or in multiple layers, for example allowing each layer to cure before application of a further layer. The application of more than one coating can be used to shield the composition from the metal container or device and prevent adhesion of the active ingredients on the container or device walls.

The term “topical composition” in some embodiments can include the sprayable liquid composition on a surface, e.g., a skin surface, which forms when the aerosol spray comes in contact with the surface. In some embodiments, administration by aerosol spray provides a more consistent administration of the composition on the subject being treated. In some embodiments, administration by aerosol spray provides a more thorough administration of the composition to the subject, e.g., more area is covered. In some embodiments, administration by aerosol spray provides a more convenient method of administration. In some embodiments, administration by aerosol spray reduces the need for other applicators, e.g., wipes, gauzes, clothes, etc. In some embodiments, administration by aerosol spray reduces exposure of the hands or other unintended body parts to the presence of spironolactone or canrenone that may occur by administration by creams, gels, or liquids. In some embodiments, administration by aerosol spray provides a more convenient mode of administration, as it allows the composition to dry quickly after application.

In some embodiments, the present disclosure provides a film composition comprising spironolactone, octisalate, butyl methacrylate and methyl copolymer (3:1), and polyethylene glycol 400 dried on the skin after application to the skin. In some embodiments, the film composition comprises about 3% w/w to about 70% w/w spironolactone. In some embodiments, the composition comprises about 20% w/w to about 50% w/w spironolactone. In some embodiments, the composition comprises about 3% w/v to about 60% w/v octisalate. In some embodiments, the composition comprises about 20% w/v to about 60% w/v octisalate. In some embodiments, the composition comprises about 3% w/v to about 60% w/v butyl methacrylate and methyl copolymer (3:1). In some embodiments, the composition comprises about 20% w/v to about 60% w/v methacrylic acid and methyl methacrylate copolymer 1:2

In embodiments in which the spray container is a pressurized device, the pressure in the sealed and pressurized device can be any pressure suitable for delivery of the sprayable liquid composition. In some embodiments, the pressure is about 28 psi to about 145 psi at 25° C.

In some embodiments, the spray container comprising the sprayable liquid composition is a misting device, with a means for providing a mist of the topical composition. As used herein, the misting device would include any device which is capable of producing fine mist particles of the sprayable liquid composition. In some embodiments, the misting device produces mist particles of the sprayable liquid composition with an average diameter of about 20 μm to about 150 μm.

### Methods of Treatment

In some embodiments, the disclosure provides a method of treating acne, male and female pattern hair loss, hirsutism, hidradenitis suppurativa, polycystic ovary syndrome (PCOS), or combinations thereof in a subject, the method comprising topically applying any of the sprayable liquid compositions described herein as a spray to a skin surface of the subject.

The sprayable liquid compositions described herein can be used in the treatment of acne, male and female pattern hair loss, hirsutism, hidradenitis suppurativa, polycystic ovary syndrome (PCOS), or combinations thereof in a subject. In some embodiments, the sprayable liquid compositions described herein can be used in the treatment of polycystic ovary syndrome (PCOS). In some embodiments, the subject is a human. In some embodiments, the human is a female. In some embodiments, the human is a male. When administered orally, spironolactone can cause adverse effects in males and females. The present disclosure provides a sprayable liquid composition of spironolactone or canrenone that can provide fewer adverse events and can be well suited for both female and male subjects who currently have fewer options regarding the management of their acne, male and female pattern hair loss, hirsutism, PCOS, or hidradenitis suppurativa.

The term “treatment” as used herein refers to reducing the severity of a symptom associated with acne, male and female pattern hair loss, hirsutism, hidradenitis suppurativa, PCOS, or combinations thereof. Thus, in some embodiments, methods provided herein provide palliative care for acne, male and female pattern hair loss, hirsutism, hidradenitis suppurativa, PCOS, or combinations thereof. In some embodiments, the term “treatment” refers to eliminating a symptom associated with acne, male and female pattern hair loss, hirsutism, hidradenitis suppurativa, PCOS, or combinations thereof. Thus, in some embodiments, the methods provided herein provide curative care associated with acne, male and female pattern hair loss, hirsutism, hidradenitis suppurativa, PCOS, or combinations thereof. In some embodiments, the methods described herein reduce and/or eliminate the severity one, two, three or more than three symptoms associated with acne, male and female pattern hair loss, hirsutism, hidradenitis suppurativa, PCOS, or combinations thereof.

The term “subsequent” as used herein refers to applications of the sprayable liquid compositions that follow or come after another application in time, order, or place. For example, an application of the sprayable liquid composition that is 24 hours after the first application of the composition to the skin is a subsequent application.

In some embodiments, the subsequent application is applied one hour to one week after the first application. In some embodiments, the subsequent application is applied two hours to 4 days after the first application. In some embodiments, the subsequent application is applied four hours to two days after the first application. In some embodiments, the subsequent application is applied six hours to one day after the first application. In some embodiments, the subsequent application is applied twelve hours to one day after the first application. In some embodiments, the subsequent application is applied “as needed” or upon the occurrence of a symptom associated with acne, male and female pattern hair loss, hirsutism, hidradenitis suppurativa, PCOS, or combinations thereof.

In some embodiments, the method of treating acne, male and female pattern hair loss, hirsutism, hidradenitis suppurativa, PCOS, or combinations thereof comprises applying the composition described herein once within a 24 hour period. In some embodiments, the method of treating acne, male and female pattern hair loss, hirsutism, hidradenitis suppurativa or PCOS, comprises applying a first application at a first timepoint and a second application at a second timepoint, wherein the second timepoint is at least 24 hours after the first timepoint. In some embodiments, the composition is applied at three or more applications at three or more timepoints. In some embodiments, the time between each timepoint is about 24 hours.

In some embodiments, the first application and the second application are applied at the same site on the skin surface of the subject. In some embodiments, the second application of the composition is applied at a site on the skin that is the site of the first application. In some embodiments, the barrier film formed on the skin from the first application of the composition is removed from the first site before the second application of the composition. In some embodiments, the barrier film formed from the first application of the composition is removed from the first site before the composition is applied to the first site in a subsequent application.

In some embodiments, the first application of the composition is applied at a site on the skin that is different from the second site. In some embodiments, the first application and the second application are applied at different sites on the skin surface of the subject.

To achieve a dosage amount sufficient for the intended therapeutic effect, it is often necessary to apply large amounts of spironolactone or canrenone compositions to large surface areas of skin. However, administration of large volumes of a drug increases the likelihood that a solution does not dry in time, and then drips in runoff from the application site, which can result in an inconsistent and inaccurate amounts delivered. Additionally, drug runoff increases the risk of transfer to other persons and unwanted side effects. Achieving ideal dosing concentrations and volumes is critical to achieving consistent and accurate dosage amount in a patient and preventing transfer to others. In some embodiments, the present disclosure provides a composition for which the required volume of composition administered is reduced, and provides a film forming layer which reduces transference.

The sprayable liquid compositions described herein can be applied in a volume of about 500 μL or less in a single actuation. In some embodiments, the composition is applied in a volume of about 300 μL or less in a single actuation. In some embodiments, the composition is applied in a volume of about 250 μL or less in a single actuation. In some embodiments, the composition is applied in a volume of about 100 μL or less in a single actuation. In some embodiments, the composition is applied in a volume of about 50 μL to about 300 μL to the skin in a single actuation. In some embodiments, the composition is applied in a volume of about 100 μL in a single actuation. In some embodiments, the composition is applied in a volume of about 50 μL in a single actuation.

The sprayable liquid compositions provided herein form a protective, washable and breathable film and thus, does not need to be covered after application to the skin. In some embodiments, the sprayable liquid composition is applied to exposed parts of the body, e.g., the arms. In some embodiments, the sprayable liquid composition is applied to an area of the skin that does need to be covered with clothing or occlusive items such as bandages.

In some embodiments, the present disclosure provides for sprayable liquid compositions that require a reduced frequency of administrations for treatment of conditions described herein. For example, in some embodiments, the composition is administered not more than once in a 24 hour period to alleviate symptoms of the conditions described herein.

All references cited herein, including patents, patent applications, papers, textbooks and the like, and the references cited therein, to the extent that they are not already, are hereby incorporated herein by reference in their entirety.

### EXAMPLES

### Example 1—Developing Sprayable Liquid Compositions of Spironolactone

Compositions 1-7 in Table 1 and Compositions 13 and 14 in Table 2 comprising spironolactone were prepared according to the manufacturing process described herein. Compositions 8-12 and 14 in Table 2 and Compositions 15-21 in Table 3 are prepared according to the manufacturing process described herein. To prepare the compositions, acetone, di-isopropyl adipate, dimethyl isosorbide, dimethyl sulphoxide, ethyl acetate, ethanol, or isopropyl alcohol were mixed in a suitable vessel to produce a solvent mixture. The film forming excipients, methacrylic acid and methyl methacrylate copolymer 1:1, methacrylic acid and methyl methacrylate copolymer 1:2, poly(butylmethacrylate-co-(2-dimethylaminoethyl)methacrylate-co-methyl methacrylate 1:2:1, hypromellose, hydroxypropyl cellulose, ethyl cellulose, polyvinylpyrrolidone, or polyvinyl acetate and polyethylene glycol 400 were added to the solvent mixture, and the mixture was stirred until a clear solution formed. The penetration enhancers, 1-dodecylazacycloheptan-2-one, isopropyl myristate, octisalate, oleic acid, or diethylene glycol monoethiyl ether (Transcutol® P), and/or a fragrance were then added to the mixture and the mixture was further stirred. The active ingredient spironolactone was then added to the mixture and the mixture was again stirred until a clear solution was achieved. Lastly, the solution was added to a suitable container and a spray pump was affixed to the container. Exemplary spironolactone spray compositions are shown below.

TABLE 1

| | Composition | | | | | | |
|---|---|---|---|---|---|---|---|
| | 1 | 2 | 3 | 4 | 5 | 6 | 7 |
| Ingredient | % w/w | | | | | | |
| Spironolactone | 1.0 | 2.0 | 3.0 | 4.0 | 6.0 | 8.0 | 10.0 |
| Ethyl Acetate USP | 30.0 | 50.5 | 47.9 | 51.0 | 50.0 | 45.0 | 46.1 |
| Di-isopropyl adipate USP | 30.0 | 14.5 | 12.4 | 10.5 | 10.5 | 10.0 | 10.5 |
| Ethanol USP | 5.0 | 5.0 | 5.0 | 5.0 | 5.0 | 5.0 | 5.0 |
| Dimethyl sulfoxide USP | 5.0 | 5.0 | 5.0 | 5.0 | 5.0 | 5.0 | 5.0 |
| Water USP | 5.0 | 5.0 | 5.0 | 5.0 | 5.0 | 5.0 | 5.0 |
| Iso-propyl Myristate | 6.0 | 6.0 | 6.0 | 6.0 | 6.0 | 6.0 | 6.0 |
| Oleic Acid USP | 4.0 | 6.0 | 7.0 | 5.0 | 6.0 | 7.0 | 6.0 |
| Eudragit ® S100 USP/NF | 7.0 | 5.0 | 7.0 | 7.0 | 5.0 | 7.0 | 5.0 |
| Polyethylene glycol 400 | 1.5 | 1.0 | 1.2 | 1.5 | 1.0 | 1.5 | 0.9 |
| Fragrance | 0.5 | 0.0 | 0.5 | 0.0 | 0.5 | 0.5 | 0.5 |
| TOTAL | 100 | 100.0 | 100.0 | 100.0 | 100.0 | 100.0 | 100.0 |

TABLE 2

| | Composition | | | | | | |
|---|---|---|---|---|---|---|---|
| | 8 | 9 | 10 | 11 | 12 | 13 | 14 |
| Ingredient | % w/w | | | | | | |
| Spironolactone | 2.0 | 4.0 | 6.0 | 6.66 | 8.0 | 20.0 | 10.00 |
| Ethyl Acetate | 45.0 | 44.4 | 40.9 | 43.84 | 43.7 | 31.5 | 42.1 |
| Di-isopropyl adipate | 20.5 | 19.1 | 17.4 | 15.5 | 15.8 | 15.5 | 15.5 |
| Dimethyl sulfoxide USP | 5.0 | 5.0 | 5.0 | 5.0 | 5.0 | 5.0 | 5.0 |
| Water USP | 5.0 | 5.0 | 5.0 | 5.0 | 5.0 | 5.0 | 5.0 |
| Dimethyl- isosorbide | 10 | 10 | 10 | 10 | 10 | 10 | 10 |
| Transcutol ® P | 4.0 | 6.0 | 7.0 | 5.0 | 6.0 | 4.0 | 6.0 |
| Eudragit ® S100 | 7.0 | 5.0 | 7.0 | 7.0 | 5.0 | 7.0 | 5.0 |
| Polyethylene glycol 400 | 1.5 | 1.0 | 1.2 | 1.5 | 1.0 | 1.5 | 0.9 |
| Fragrance | 0.0 | 0.5 | 0.5 | 0.5 | 0.5 | 0.5 | 0.5 |
| TOTAL | 100.0 | 100.0 | 100.0 | 100.0 | 100.0 | 100.0 | 100.0 |

TABLE 3

| | Composition | | | | | | |
|---|---|---|---|---|---|---|---|
| | 15 | 16 | 17 | 18 | 19 | 20 | 21 |
| Ingredient | % w/w | | | | | | |
| Spironolactone USP | 1.0 | 2.0 | 4.0 | 6.00 | 8.00 | 10.00 | 20.00 |
| Ethyl Acetate USP | 36.0 | 38.5 | 34.9 | 34.5 | 35.5 | 30.0 | 10.0 |
| Di-isopropyl adipate USP | 30.0 | 29.5 | 27.4 | 25.5 | 25.5 | 26.0 | 25.5 |
| Dimethyl sulfoxide USP | 5.0 | 5.0 | 5.0 | 5.0 | 5.0 | 5.0 | 5.0 |
| Water USP | 5.0 | 5.0 | 5.0 | 5.0 | 5.0 | 5.0 | 5.0 |
| Oleic Acid USP | 15.0 | 15.0 | 15.0 | 15.0 | 15.0 | 15.0 | 25.0 |
| Eudragit ® S100 USP/NF | 7.0 | 4.0 | 7.0 | 7.0 | 5.0 | 7.0 | 7.0 |
| Polyethylene glycol 400 | 1.5 | 1.0 | 1.2 | 1.5 | 1.0 | 1.5 | 2.0 |
| Fragrance | 0.5 | 0.0 | 0.5 | 0.5 | 0.0 | 0.5 | 0.5 |
| TOTAL | 100.0 | 100.0 | 100.0 | 100.0 | 100.0 | 100.0 | 100.0 |

### Example 2—Dual Chamber Spironolactone Spray Compositions

Exemplary compositions for a dual chamber dispensing systems are outlined in Table 4 below. Solutions from both chambers are kept separate until mixing during application either by concomitant actuation of the sprays from each of the chambers or by application from a pad when a seal is broken between pouches containing each formulation.

TABLE 4

| | Composition | | | | | | |
|---|---|---|---|---|---|---|---|
| | 22 | 23 | 24 | 25 | 26 | 27 | 28 |
| | % w/w | | | | | | |
| Ingredients (Chamber 1) | | | | | | | |
| Spironolactone USP | 0.6 | 0.6 | 1.2 | 1.7 | 2.0 | 3.0 | 5.0 |
| Ethylacetate USP | 19.1 | 19.1 | 19.1 | 15.4 | 21.2 | 15.7 | 15.5 |
| Di-isopropyl adipate USP | 10.0 | 10.0 | 10.0 | 10.0 | 10.0 | 10.0 | 10.0 |
| Ingredients (Chamber 2) | | | | | | | |
| Ethanol USP | 58.1 | 58.2 | 54.2 | 59.4 | 54.7 | 55.9 | 56.9 |
| Octisalate USP | 4.0 | 6.0 | 7.0 | 5.0 | 6.0 | 7.0 | 6.0 |
| Eudragit ® S100 USP/NF | 7.0 | 5.0 | 7.0 | 7.0 | 5.0 | 7.0 | 5.5 |
| Polyethylene Glycol 400 | 1.2 | 1.1 | 1.5 | 1.5 | 1.1 | 1.4 | 1.1 |
| TOTAL | 100.0 | 100.0 | 100.0 | 100.0 | 100.0 | 100.0 | 100.0 |

### Example 3-Spironolactone Spray Compositions with a Peelable Film

Compositions 33 and 34 in Table 5 comprising spironolactone were prepared according to the manufacturing process described herein. Compositions 29-32 in Table 5 are prepared according to the manufacturing process described herein. To prepare the compositions, di-isopropyl adipate and water were mixed in a suitable vessel with the film forming excipients polyvinylpyrrolidone and polyvinyl acetate. Polyethylene glycol 400 was added to the mixture, and the mixture was stirred until a clear solution formed. The penetration enhancer octisalate was then added to the mixture, and the mixture was further stirred. The active ingredient spironolactone was then added to the mixture and the mixture was again stirred until a clear solution was achieved. Lastly, the solution was added to a suitable container and a spray pump was affixed to the container.

TABLE 5

| | Composition | | | | | |
|---|---|---|---|---|---|---|
| | 29 | 30 | 31 | 32 | 33 | 34 |
| Ingredient | % w/w | | | | | |
| Spironolactone USP | 0.1 | 0.1 | 0.1 | 0.2 | 0.2 | 0.2 |
| Di-isopropyl adipate | 58.6 | 58.8 | 55.4 | 61.1 | 62.7 | 59.2 |
| USP | | | | | | |
| Water USP | 5.0 | 5.0 | 5.0 | 5.0 | 5.0 | 5.0 |
| Octisalate USP | 4.0 | 6.0 | 7.0 | 5.0 | 6.0 | 7.0 |
| Polyvinylpyrrolidone | 3.0 | 5.0 | 4.0 | 7.0 | 5.0 | 7.0 |
| and Polyvinylacetate | | | | | | |
| Copolymer USP/NF | | | | | | |
| Polyethylene Glycol 400 | 1.2 | 1.0 | 1.4 | 1.5 | 0.9 | 1.4 |
| TOTAL | 100.0 | 100.0 | 100.0 | 100.0 | 100.0 | 100.0 |

### Example 4-Spironolactone Spray Compositions with an Antioxidant

Exemplary spironolactone spray compositions containing niacinamide shown in Table 6.

TABLE 6

| | Composition | | | | | |
|---|---|---|---|---|---|---|
| | 35 | 36 | 37 | 38 | 39 | 40 |
| Ingredient | % w/w | | | | | |
| Spironolactone USP | 5.7 | 5.7 | 5.7 | 10.0 | 10.0 | 10.0 |
| Ethyl Acetate USP | 53.1 | 47.2 | 49.7 | 49.3 | 43.7 | 47.4 |
| Di-isopropyl adipate USP | 20.0 | 20.0 | 19.0 | 10.0 | 10.0 | 10.0 |
| Water USP | 5.0 | 5.1 | 5.2 | 5.2 | 10.2 | 11.2 |
| Octisalate USP | 4.0 | 6.0 | 7.0 | 5.0 | 6.0 | 7.0 |
| Eudragit ® S100 USP/NF | 3.0 | 5.0 | 3.5 | 7.0 | 5.0 | 4.0 |
| Polyvinylpyrrolidone | 3.0 | 5.0 | 3.5 | 7.0 | 5.0 | 4.0 |
| USP/NF | | | | | | |
| Polyethylene Glycol 400 | 1.2 | 1.0 | 1.4 | 1.5 | 5.1 | 1.4 |
| Niacinamide | 5 | 5 | 5 | 5 | 5 | 5 |
| TOTAL | 100.0 | 100.0 | 100.0 | 100.0 | 100.0 | 100.0 |

### Example 5—Analysis of Skin Permeation of Spironolactone Film Forming Compositions

The degree of permeation of spironolactone film forming compositions into skin were tested. Franz diffusion cells with a donor area of 4.9 cm² and 20 mL receptor chamber volume were assembled with excised abdominal porcine skin. The receptor chamber was filled with 0.1 M sodium citrate buffer to maintain the pH around 4.5. For each experiment, 60 μl of either 3% w/w or 5% w/w spironolactone spray film forming composition (Compositions 27 and 28 of Table 4) was added to the donor chamber of the cell.

The samples were maintained at 37° C. and the receptor chamber solution was magnetically stirred to maintain homogeneity. Samples of receptor medium were collected at 1, 2, 4, 6, and 24 hour timepoints post addition of the spironolactone compositions to the cells and then analyzed by HPLC to determine the amount of spironolactone permeated through the skin layers.

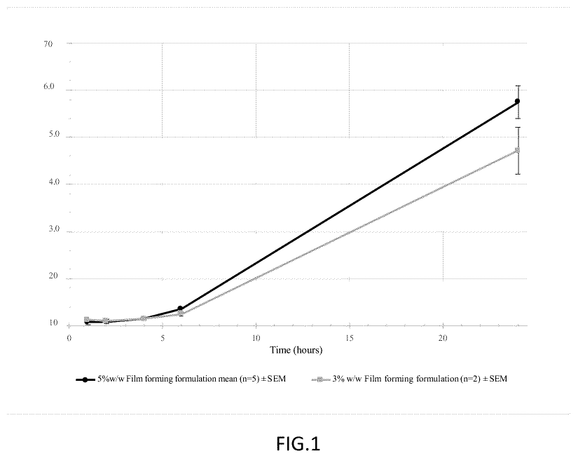

As shown in FIG. 1, spironolactone permeated through the skin in less than 2 minutes for both the 3% w/v and 5% w/v spironolactone film forming compositions. The permeation profiles of both compositions show that spironolactone permeated through the skin over 24 hours.

### Example 6—Analysis of Film Breathability of the Spironolactone Film Forming Compositions

The film formed after application of the spironolactone composition is designed to remain on the skin for at least 24 hours. It is desirable that moisture vapor is able to pass through the film. A breathable film ensures that the underlying skin remains in good condition, preventing the development of erythema and other adverse local reactions to the skin. The breathability of the film was tested using the procedure below.

The 5% w/v spironolactone film forming composition (Composition 28 of Table 4) sample film was applied to the surface of porous surgical tape (Transpore™, 3M, St. Paul, MN) membrane at 12.5 μl per cm² and allowed to dry. A water vapor impermeable container with an opening at the top was partially filled with water. The composition was applied to the porous tape, and the tape with the composition applied was placed over the opening of the container. The container was stored at 37° C. in the presence of calcium chloride desiccant, which maintained a low humidity within the chamber. Water loss from the container over time was determined gravimetrically by measuring the container weight over a period of at least 48 hours. A water vapor transmission rate per area was then determined. Two reference control evaluations were also used in this experiment, a non-occluded (non-film forming) control applied to only the porous tape, and an occluded control applied to porous tape which was covered with non-porous packaging tape. Both controls were applied to the surface of porous surgical tape (Transpore™, 3M, St. Paul, MN) membrane at 12.5 μl per cm2 and allowed to dry.

The relative transmission was derived from equation

$$\frac{( Sample Mean Rate - Occluded Control Mean Rate )}{( \begin{matrix}Non - occluded Control Mean Rate - \\ Occluded Control Mean Rate\end{matrix} )} .$$

The sample spironolactone film had a mean water vapor transmission rate of 1743 g/m² per 24 hours. The rate of mean water vapor transmission as a fraction relative to the non-occluded control was 0.80.

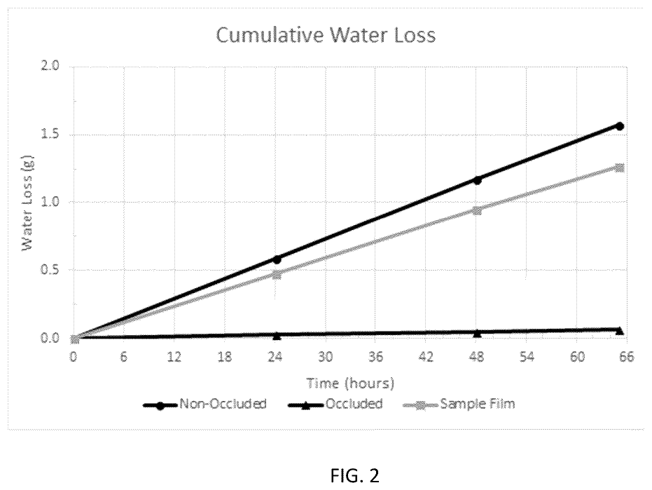

The high-water vapor transmission rate relative to the non-occluded control indicates that the spironolactone film is not obstructing water vapor transmission and thus maintains the skin in healthy condition. The rate of water loss for each composition over a period of 48 hours is shown in FIG. 2.

### Example 7—Analysis of Drug Transfer of the Spironolactone Film Forming Compositions

A skin contact transfer study was conducted to compare the active ingredient transfer from the skin after application of a 10% spironolactone film forming spray composition (Composition 7 from Table 1) and a non-occluded 10% spironolactone gel composition. Each composition was applied to a glass coupon and allowed to dry for 15 minutes. A polyester swab was firmly pressed against the application area on the coupon. The swab was moved back and forth in overlapping passes over the entire area. The swab passes were repeated alternating the coupon orientation and swab side covering the area for a total of 8 times. The residue on the swab was extracted in 10 mL ethanol and the recovered solutions were analyzed by HPLC. The recovered amounts of spironolactone were calculated by comparison to a standard of known concentrations.

The transferred residues for the spironolactone film forming composition were 0.5±0.3% (mean±SD, n=6) of the applied dose. The transferred residues for the spironolactone gel composition were 84.0±9.0% (mean±SD, n=6) of the applied dose. These results indicate that the spironolactone sprayable liquid compositions forms an effective barrier to secondary transfer. The film produced acts in a similar manner to a clothing barrier to prevent drug transfer.

Additional tests were performed to test the effectiveness of the barrier film. A second application of the film forming composition was sprayed on top of the previously applied spray comprising the formed barrier film. There was no permeation of spironolactone from the second application of the spray through the barrier film and to the underlying skin. These results indicate that the barrier film is effective in preventing movement of the active ingredient or access to the skin underneath.

### Example 8—Analysis of Film Washability of the Spironolactone Film Forming Compositions

Washability studies were conducted to determine the washability of the film formed after application of the film forming composition to the skin. The 10% spironolactone film forming composition (Composition 7 of Table 1) was applied to excised porcine abdominal skin and allowed to dry for 15 minutes. The skin was washed by wiping with a wet soft sponge that was periodically dipped in warm water for 1 minute. The skin was then swabbed with an alcohol wipe. An unwashed control was prepared by wiping the skin surface with only an alcohol wipe to remove the film without washing. The residual spironolactone on the wipes was quantified.

Recovery of spironolactone from the wipe used after washing the skin surface with water showed that 0.5% of the applied dose was recovered from the skin surface. Recovery of spironolactone from the wipe used on the unwashed control sample showed that 84% of the applied dose was recovered from the wipe. These results indicate that the film formed post application of the composition is effectively removed from the skin via washing with water.

### Example 9—Analysis of Crystallization in Film Forming Composition

The _spironolactone film forming composition (Composition 7 of Table 1) was evaluated for any crystallization of spironolactone. The _% spironolactone composition was prepared according to the manufacturing process as described herein. The compositions were then analyzed visually under the microscope for evidence of spironolactone crystals.

| Composition | Observation |
|---|---|
| 10% spironolactone film forming | No crystals formed |
| composition | |

1. A sprayable liquid composition comprising:
a. about 1% w/v to about 20% w/v spironolactone or canrenone;
b. about 30% w/v to about 97% w/v aliphatic solvent;
c. about 1% w/v to about 10% w/v water;
d. one or more film forming excipients, wherein the film forming excipient has a solubility in water at a pH between 1 and 10, and
e. a penetration enhancer,
wherein the composition forms a washable and/or a peelable film when sprayed on a skin surface, and wherein the spironolactone does not crystallize when the composition is applied to the skin surface.

2-4. (canceled)

5. The composition of claim 1, wherein the aliphatic solvent is acetone, di-isopropyl adipate, dimethyl isosorbide, dimethyl sulphoxide, ethyl acetate, ethanol, isopropyl alcohol, or combinations thereof.

6. (canceled)

7. (canceled)

8. The composition of claim 1, wherein the aliphatic solvent is
a. about 15% w/v to about 60% w/v ethyl acetate;
b. about 10% w/v to about 65% w/v di-isopropyl adipate; and
c. about 5% w/v to about 60% w/v ethanol.

9. The composition of claim 1, wherein the film forming excipient comprises a polyacrylate polymer, a vinylpyrrolidone polymer, methacrylic acid and methyl methacrylate copolymer 1:1, methacrylic acid and methyl methacrylate copolymer 1:2, poly(butylmethacrylate-co-(2-dimethylaminoethyl)methacrylate-co-methyl methacrylate 1:2:1, hypromellose, hydroxypropyl cellulose, ethyl cellulose, butyl methacrylate and methyl copolymer (3:1), polyvinylpyrrolidone, polyvinylpyrrolidone, polyvinyl acetate or combinations thereof.

10. (canceled)

11. The composition of claim 1, wherein the film forming excipient is about 1% to about 10% w/v of the composition.

12. The composition of claim 1, wherein the penetration enhancer is azone, isopropyl myristate, octisalate, oleic acid, Transcutol® P or combinations thereof.

13. (canceled)

14. The composition of claim 1, wherein the penetration enhancer is about 1% to about 10% w/v of the composition.

15. (canceled)

16. The composition of claim 1, wherein the composition further comprises a washability enhancer, and wherein the washability enhancer is polyethylene glycol 400.

17. (canceled)

18. The composition of claim 1, wherein the composition further comprises a buffer, and wherein the buffer comprises sodium citrate, citric acid buffer, or combinations thereof.

19. (canceled)

20. The composition of claim 1, wherein the composition further comprises an additional active agent, and wherein the additional active agent is minoxidil, finasteride, dutasteride, niacinamide or combinations thereof.

21. (canceled)

22. (canceled)

23. The composition of claim 20, wherein the additional active agent is
a. about 1% w/w to about 10% w/w minoxidil;
b. about 0.1% w/w to about 1.0% w/w finasteride or dutasteride;
c. about 1% w/w to about 10% w/w minoxidil and about 0.1% w/w to about 1.0% w/w finasteride or dutasteride; or
d. about 2% w/v to about 10% w/v niacinamide.

24. The composition of claim 1, wherein the pH of the composition is about 4 to about 5.

25. (canceled)

26. The composition of claim 1, wherein the composition forms a barrier film less than three minutes after application to the skin surface.

27. (canceled)

28. The composition of claim 1, wherein the composition has a viscosity of less than 30 cPs at room temperature.

29. The composition of claim 1, wherein the composition has a water vapor transmission rate, as a fraction compared to a non-occluded control, not less than 0.50 over a 48-hour period when applied and dried on a porous substrate.

30. A spray container comprising
a. the composition of claim 1; and
b. a metering valve.

31. The spray container of claim 30, wherein the container further comprises a dose indicator.

32. (canceled)

33. The spray container of claim 30, wherein the container further comprises a propellent.

34-40. (canceled)

41. A method of treating acne, male and female pattern hair loss, hirsutism, hidradenitis suppurativa, polycystic ovary syndrome (PCOS), or combinations thereof in a subject, the method comprising topically applying the composition of claim 1 as a spray to a skin surface of the subject.

42-55. (canceled)

56. A dual chamber device comprising:
a. a first chamber comprising a first composition comprising 1% w/w to 10% w/w spironolactone, about 15% w/w to 20% w/w ethyl acetate and about 10% di-isopropyl adipate;
b. a second chamber comprising a second composition comprising about 50% w/w to about 60% w/w ethanol, about 4% to 7% octisalate, about 5% w/v to about 7% w/v butyl methacrylate and methyl copolymer (3:1) and about 1.5 w/w polyethylene glycol 400,
wherein the dual chamber device is suitable for providing a spray comprising the composition.

57. (canceled)

58. (canceled)
